(19) United States
(12) Patent Application Publication
(10) Pub. No.: US 2026/0041637 A1
(43) Pub. Date: Feb. 12, 2026

(54) VESICLES BASED ON GLUCOSE-DERIVED SURFACTANTS AND PHYTOSTEROLS

(71) Applicants: NANOMOL TECHNOLOGIES, S.L., CERDANYOLA DEL VALLÈS (ES); CONSEJO SUPERIOR DE INVESTIGACIONES CIENTÍFICAS (CSIC), MADRID (ES)

(72) Inventors: ALBA CÓRDOBA INSENSÉ, CERDANYOLA DEL VALLÈS (ES); MARTA ALCAINA HERNANDO, CERDANYOLA DEL VALLÈS (ES); SANTIAGO SALA VERGÉS, CERDANYOLA DEL VALLÈS (ES); LEONOR VENTOSA RULL, BELLATERRA (ES); IVANA MALVACIO, CERDANYOLA DEL VALLÈS (ES)

(21) Appl. No.: 19/100,302

(22) PCT Filed: Jul. 31, 2023
(86) PCT No.: PCT/EP2023/071210

§ 371 Date: Jan. 31, 2025

(30) Foreign Application Priority Data
Aug. 3, 2022 (EP) 22382751.0

(51) Int. Cl. A61K 9/1272; A61K 9/1277; A61K 47/26; A61K 47/28; A61K 49/00

(52) U.S. Cl. CPC A61K 9/1272; A61K 9/1277; A61K 47/26; A61K 47/28; A61K 49/00

(57) ABSTRACT

A vesicle comprising at least one phytosterol and at least one glucose-derived surfactant and a composition comprising a plurality of such vesicles. The vesicle and the composition comprising a plurality of vesicles are used in the manufacture of a suspension, as a delivery system or as medicament. Further, a method for the manufacture of these vesicles.

### FIELD OF THE INVENTION

The present invention relates to the field of vesicular systems. In particular, the present invention relates to vesicles based on phytosterols and glucose-derived surfactants. It is also provided a method for obtaining vesicular systems based on DELOS-susp methodology and their applications in the cosmetic and pharmaceutical field.

### BACKGROUND ART

Different delivery systems based on nano and microtechnology have been extensively explored in the pharmaceutical industry, not only for therapeutic applications but also for bioimaging and diagnostic. Besides medical applications, vesicular systems (or vesicles, Vs) are booming the healthcare and cosmetic sector. The most widely studied and currently used systems are liposomes, which allow for the protection of sensitive molecules and a controlled permeation of the active ingredients in the target organ. Nevertheless, there are some drawbacks regarding their physicochemical properties and stability, which affect their reproducibility and limit their applications. Liposomes suffer physical alterations, i.e., instability, which lead to an increased permeability and ultimately causes undesired or rapid leakage rates. Further, liposomes also suffer chemical alterations which relate to the hydrolysis, oxidation and peroxidation of the constitutive lipid molecules. Consequently, limiting conditions, such as the addition of cholesterol, lower storage temperatures or the addition of antioxidants, are required to overcome these drawbacks and synthesize such systems, while maintaining their stability and allowing for their optimal storage.

Another kind of drug delivery system gaining scientific interest are niosomes (non-ionic surfactants vesicles), for which, although they are considered liposome analogues with higher stability, some articles report short stability due to aggregation, fusion swelling, and drug leakage problems. On the other hand, quatsomes, a particular type of Vs formed by self-assembly of cholesterol molecules and quaternary ammonium surfactants (e.g., cetyl trimethyl ammonium bromide, myristalkonium chloride, cetylpyridinium chloride, steralkonium chloride, etc.) are potential delivery systems for pharmaceutical and cosmetic ingredients because of their good physicochemical properties and long-term stability. However, the use of such surfactants in healthcare and pharmaceutical products is limited due to some reported skin irritation and other toxicological effects at high concentrations.

Consequently, a new generation of ecological and environmentally safe surfactants has started to be studied recently: glucose-derived surfactants (e.g., alkyl polyglucosides (APGs)). However, scarce examples of vesicle formation comprising this new generation of surfactants have been found in the art.

Further, it is underlined that the size and lamellarity of Vs are important structural parameters that need to be controlled, since they are crucial factors affecting the performance of vesicles, e.g., as pharmaceutical carriers. In those lines, depending on their size and lamellarity, vesicular systems can be classified into small unilamellar vesicles (SUVs, d<200 nm), large unilamellar vesicles (LUVs, d>200 nm), and multilamellar vesicles (MLVs).

Vesicle membrane also plays an important role in terms of its stability, rigidity, permeability, functionalization, and response to external stimuli. Thus, Vs performance is highly affected by their homogeneity, not only in size or morphology, but also in the membrane composition and supramolecular organization. Structural homogeneity of Vs is key to achieve uniform and reproducible delivery responses, along with an homogeneous release of the entrapped active at the site of action.

Salim et al. (2015) reported the formation of Vs by Thin Film Hydration (TFH) using octyl glucoside and octyl maltoside in combination with 20 mol % of cholesterol and 8 mol % dicetyl phosphate (DCP). The stability of the Vs was >3 months at room temperature (RT), but the storage at 4° C. triggered the formation of a white solid precipitate that may be caused by lipid-cholesterol phase separation. These systems were used for the encapsulation of methylene blue with a low encapsulation efficiency (EE) of 15-20%.

Muzzalupo et al. (2013) reported Vs formation by TFH using octyl glucoside, decyl glucoside and lauryl glucoside with cholesterol in molar ratio 1:1 in water. The stability of the system was >12 months at RT. The encapsulation of methotrexate was studied and reported with an EE of 80-95%.

Manconi, Vila et al. (2006) described the formation of multilamellar vesicles (MLVs) by TFH using decyl glucoside and cholesterol in different molar ratios, with particles sizes ranging from 1263.2 nm to 338.6 nm. MLVs prepared in this study were large and polydisperse (Pdl>0.8). The encapsulation of methylene blue was tested, obtaining an EE of 40-70%.

Manconi, Sinico et al. (2006) reported the formation of Vs by TFH in PBS of decyl glucoside: cholesterol and caprylyl/capryl glucoside: cholesterol, using dicetyl phosphate (DCP) or stearylamine as additives. Some of these systems were used to encapsulate tretinoin, a natural retinoid, which resulted in an EE of 93-99.5%. The obtained tretinoin-loaded systems were mainly polydisperse MLVs, with large particle sizes (>500 nm for saturated active concentration, and >264 nm for unsaturated active concentration). Further, only after several cycles of sonication unilamellar vesicles could be obtained.

As described above, cholesterol is widely used in the formation of Vs and, generally, in health and personal care products. Nevertheless, the increasing awareness of the negative consequences resulting from the use of animal-derived products urges the need to develop new vesicle systems comprising ecological, animal-free, and consumer-friendly components.

### SUMMARY OF THE INVENTION

One problem to be solved by the present invention is to provide a sustainable vesicle platform useful for the efficient encapsulation of agents, such as pharmaceutical and cosmetic agents.

The present invention generally relates to particles, including vesicles (Vs), vesicular systems or other micro or nanoentities, comprising phytosterols (e.g., β-sitosterol) and glucose-derived surfactants (e.g., alkyl polyglucoside surfactants). The particles developed in the present invention are stable and capable of encapsulating agents such as pharmaceutical or cosmetic agents. Thus, the present invention provides a new and highly versatile system of which are not only consumer-friendly, but also contribute to a more sustainable manufacturing.

Inventors have developed a platform that produces unilamellar and homogenous Vs based on the use of plant-derived ingredients and sugar-based surfactants by applying e.g., DELOS-susp methodology (as described in WO2006/079889 A1). In the present invention, phytosterols (e.g., β-sitosterol), instead of animal-derived sterols (e.g., cholesterol), are used as key components of the vesicle membrane. This substitution, which may be total or partial, constitutes a clear improvement over the prior art in terms of sustainability, as it is well known that animal-derived products have a significantly higher impact on environmental degradation, biodiversity loss and climate change than products which are plant-derived. Additionally, this new vesicle system is also aligned with increasingly popular practices such as veganism and other animal-free consumption, thus having the capacity to reach a higher number of subjects in need, and consequently increasing its scope of applicability.

Furthermore, the Vs of the present invention are characterized not only for the use of phytosterols, but also of glucose-derived surfactants, particularly alkyl polyglucosides (APGs). Glucose-derived surfactants constitute a new generation of green, biocompatible and biodegradable surfactants, which are commonly used in cleansing products for skin and hair care because they display dermatological and ocular safety, good wettability, excellent foaming performance and cleaning ability. The use of glucose-derived surfactants for nano and microcarriers has been previously described, however very scarcely studied and developed. It is believed that a vesicle system which simultaneously comprises phytosterols, such as β-sitosterol (Sit), and glucose-derived surfactants as an alternative to other surfactants is provided for the first time.

Surprisingly, inventors have found that Vs comprising phytosterols and glucose-derived surfactants (e.g., APGs), require different conditions to form, in respect to what has been previously disclosed. As stated by the prior art, Vs comprising animal-derived sterols (i.e., cholesterol) can be synthesized using APGs as surfactants which have a carbon chain length of eight, ten or twelve carbons (Muzzalupo et al. 2013). Nevertheless, as shown in EXAMPLE 1.2.1, only APGs with a chain length of 12 or more carbons can be used to form Vs comprising phytosterols (e.g., Sit). Therefore, the present invention shows that the type of sterol used, despite any structural similarity, determines which surfactants' chain length can lead to a successful formation of Vs. This effect of surfactant alkyl chain length in the formation of Vs has been proved herein regardless of the method of synthesis used (Thin Film Hydration (TFH) or DELOS-susp methodology). Altogether, inventors have been capable to determine and adequately perform the unexpected conditions required to synthesize this new vesicular system.

Moreover, the present invention does not only disclose a successful synthesis of such Vs, but also shows their high-quality performance in terms of encapsulation of numerous active ingredients representing different types of agents, including pharmaceutical and cosmetic molecules. As known by a skilled person in the field of delivery systems, the efficient formation of vesicles, as well as the later adequate encapsulation of active ingredients are highly challenging processes, which commonly result in unsatisfactory outcomes. These are, for instance, the lack of Vs formation, an inadequate formation of Vs which include the formation of other undesirable structures, or the formation of Vs which are then not capable of efficiently encapsulating one or more active ingredients of interest, or show poor colloidal stability. Thus, the extraordinary and versatile capability of the present Vs to encapsulate diverse active ingredients is to be considered as unexpected by the skilled in the art.

Working examples herein provide detailed experimental data demonstrating the formation of small homogeneous Vs composed of a combination of phytosterols and glucose-derived surfactants as the main building blocks, in aqueous media. The system provided herein is highly versatile allowing for the formation of neutral, positive and negatively charged Vs. Furthermore, this platform is shown herein to be able to integrate numerous active compounds of different nature, such as lipophilic and hydrophilic small actives, proteins, and dyes. Thus, showing a clear potential to become a sustainable delivery platform for healthcare and cosmetic applications, among others.

EXAMPLES 1.2.1 and 1.2.2 show the successful formation of Vs comprising phytosterols and glucose-derived surfactants (i.e., APGs), only resulting from the unexpected condition of using surfactants with a chain length of 12 or more carbons. Thus, a surprising effect of surfactant alkyl chain length in the formation of Vs is shown herein, regardless of the method used, either TFH or DELOS-susp methodology.

EXAMPLE 1.2.3 shows the synthesis of neutral, positively, and negatively charged Vs by self-assembly of phytosterols and APGs (e.g., lauryl glucoside, LGL), among other components, using DE-LOS-susp methodology. As disclosed in EXAMPLE 1.2.4, both APGs of cosmetic grade, as well as highly pure APGs (e.g., LGL 98% purity) were useful in the formation of neutral Vs. In terms of stability, over twelve months were reached by Vs containing the less pure LGL, and Vs containing highly pure LGL were also efficiently stabilized (see section 1.2.5). These results prove the broad scope of potential applications of said system in different fields, such as the pharmaceutical and the cosmetic field.

EXAMPLE 1.2.6 also shows the high versatility of the herein provided system, through the synthesis of both negatively and positively charged Vs. A successful formation of Vs was achieved with three anionic additives: lauryl glucoside carboxylate (LGC), sodium laurate (SL) and sodium lauroyl sarcosinate (SLS), as well as DC-cholesterol as cationic additive. All forms of Vs showed a significantly high stability of from over six months up to over a year. These results confirm, not only the versatility of the present platform, but also its consistency.

EXAMPLE 2 shows that the Vs of the present invention have an outstanding efficiency to encapsulate different active ingredients. Negatively charged Vs were capable to efficiently encapsulate lipophilic small molecules (e.g., 7-dehydrocholesterol, tocopherol and cannabidiol), as well as small proteins with over 99% of efficiency. On the other side, positively charged Vs were capable to very highly efficiently encapsulate both lipophilic and hydrophilic actives, with an unexpected efficiency of between 88 and >99%. Again, these results evidence the exceptional capability of the present vesicle system to encapsulate different types of actives ingredients due to its high versatility, and consequently proving its broad scope of potential applications. It is worth noting that no prior art documents which disclose the synthesis of similar Vs systems have disclosed the ability to efficiently encapsulate such diversity of active ingredients.

EXAMPLE 3 shows specific activity assays for two different active molecules tested after being encapsulated in the new Vs developed. The activity of the free versus encapsulated actives is compared to demonstrate that the molecules maintain their properties when loaded in the Vs.

Particularly, EXAMPLE 3.2.1 shows that the antioxidant capacity of tocopherol when dissolved in ethanol is similar to the one shown when the active is encapsulated in the negatively charged Vs composed by Sit, LGL 98% purity and LGC.

EXAMPLE 3.2.2 shows that 7-dehydrocholesterol is able to transform by light irradiation to cholecalciferol (Vitamin D) after encapsulation in the Vs. Cholecalciferol is a vitamin which plays a role in biochemical pathways of different types of cells. The example shows that the capacity of 7-dehydrocholesterol to be transformed to Vitamin D form after encapsulation in the Vs is maintained, and in the same conversion ratio observed when a suspension of the free active is irradiated.

Altogether, inventors have developed a new sustainable system of vesicles comprising phytosterols and glucose-derived surfactants which exhibit highly desirable results in terms of composition and physicochemical properties and are capable to encapsulate active ingredients of interest of different typologies, such as proteins or small hydrophilic/lipophilic molecules. Therefore, the present invention appears as a good candidate system of Vs to be applied in relevant fields such as the pharmaceutical and cosmetic field.

Accordingly, a first aspect of the invention relates to a vesicle comprising at least one phytosterol and at least one glucose-derived surfactant.

A vesicle or a vesicular system comprises an enclosed aqueous liquid compartment separated from its surroundings by one or more lipid bilayers.

A second aspect of the invention relates to a composition comprising a plurality of vesicles as defined herein.

In another aspect, the invention relates to a pharmaceutical composition comprising a therapeutically effective amount of Vs as defined herein and at least one pharmaceutically acceptable excipient, vehicle or carrier.

In another aspect, the invention relates to a cosmetic composition comprising a cosmetically effective amount of vesicles as defined herein and at least one cosmetically acceptable excipient, vehicle or carrier.

In another aspect, the invention relates to a nutraceutical composition (e.g., a food supplement) comprising a nutraceutically effective amount of vesicles as defined herein and at least one nutraceutically acceptable excipient, vehicle or carrier.

In another aspect, the invention relates to a food composition/food additive comprising an effective amount of vesicles as defined herein and at least an acceptable excipient, vehicle or carrier.

In another aspect, the invention relates to a detecting or diagnostic composition comprising an effective amount of vesicles as defined herein and at least an acceptable excipient, vehicle or carrier.

Another aspect of the invention relates to the vesicles or a composition comprising vesicles as defined herein (particularly a pharmaceutical composition), for use as a medicament.

Other aspects of the invention relate to use of the vesicles or a composition comprising vesicles as defined herein, in the manufacture of a suspension, as a delivery system or as a diagnosing or detecting tool.

Finally, another aspect of the invention relates to a method for the production of vesicles, as described herein, using the DELOS-susp methodology.

Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. Furthermore, the present invention covers all possible combinations of particular and preferred embodiments described herein. The following examples and drawings are provided herein for illustrative purposes, and without intending to be limiting to the present invention.

### DESCRIPTION OF DRAWINGS

FIG. 1 shows a scheme of the procedure of the preparation of vesicles by DELOS-susp technology, including: (A) addition of the organic solution; (B) Expansion: addition of compressed CO₂ and formation of a CO₂-expanded solution; and (C) depressurization into an aqueous solution. “OP” corresponds to “organic phase”, “AP” to “aqueous phase”, “ML” to “membrane lipids”, “MS” to “membrane surfactants”, “HOA” to “hydrophobic actives” and “HIA” to “hydrophilic actives”.

FIG. 2 shows the molecular structures of sterols and surfactants used for the synthesis of the vesicle platform. “MBB” corresponds to “main building blocks”; “CMC” to “charge membrane components” and “A” to “additives”.

FIG. 3 shows an analysis of Sit: OGL-p, Sit: DGL-p and Sit: LGL-p Vs by (A) macroscopic appearance; (B) microscopic images taken with Morphology G3 optical microscope at 50X; (C) cryo-TEM images of general views of the grid (top images, 50X) and focused on Vs (bottom images, 3000X) and (D) comparison of the samples by DLS. “S” corresponds to “Size”, and “I” corresponds to “Intensity”.

FIG. 4 shows the representations of the composition of the Vs obtained by DELOS technology: (A) Sit: LGL; (B) Sit: LGL: Chol-PEG600; (C) Sit: LGL: anionic additive (1:1:0.05-0.1); (D) Sit: LGL: anionic additive (1:0-0.8:0.2-1); (E) Sit: LGL: cationic additive (1:1:0.05); (F) Sit: LGL: cationic additive (0-0.8:1:0.2-1). “NEG” corresponds to “negatively charged molecule” and “POS” corresponds to “positively charged molecule”.

FIG. 5 shows representative cryo-TEM images of neutral Sit: LGL Vs prepared using LGL surfactants of different quality, including: (A) Sit: LGL-mc12-16 (1:0.75); (B) Sit: LGL-mc12-16 (1:1); (C) Sit: LGL (LGL-p) (1:1). (D) shows a comparison of the three samples by DLS. “S” corresponds to “Size”, and “I” corresponds to “Intensity”.

FIG. 6 shows (A) representative cryo-TEM image of Sit: LGL-p: Chol-PEG600 Vs. (B) representation of particle size by DLS. “S” corresponds to “Size”, and “I” corresponds to “Intensity”. In this figure, LGL is LGL-p.

FIG. 7 shows representative cryo-TEM images of Sit: LGL Vs stabilized with ionic additives, including (A) Sit: LGL: LGC (1:1:0.05); (B) Sit: LGL: SL (1:1:0.05); (C) Sit: LGL: SLS (1:1:0.05); (D) Sit: LGL: DC-Chol (1:1:0.05). (E) shows a comparison of the samples by DLS. “S” corresponds to “Size”, and “I” corresponds to “Intensity”. In this figure, LGL is LGL-p.

FIG. 8 shows (A) a representative cryo-TEM image of Sit: LGL: DC-Chol (0:1:1) and (B) the physicochemical characterization of positively charged Sit: LGL: DC-Chol Vs in terms of medium size(S), ζ-Potential (ζ-Pot) and Polydiversity Index (Pdl). In this figure, LGL is LGL-p.

FIG. 9 shows (A) a representative cryo-TEM image of Sit: LGL: LGC (1:0.8:0.2 and 1:0:1) and (B) the physicochemical characterization of positively charged Sit: LGL: LGC Vs in terms of medium size(S), ζ-Potential (ζ-Pot) and Polydiversity Index (Pdl). In this figure, LGL is LGL-p.

FIG. 10 shows the comparison of Sit: LGC active-loaded formulations in terms of particle size(S), ζ-Potential (ζ-Pot), Polydiversity Index (Pdl) and encapsulation efficiency (EE). 7-DHC, CBD, Dil/DID, Dil, DID, NCA, AG, b-FGF are referred in Example 2.1.

FIG. 11 shows the comparison of Sit: LGL: LGC active-loaded formulations in terms of particle size(S), ζ-potential (ζ-Pot), Polydiversity Index (Pdl) and encapsulation efficiency (EE). 7-DHC, CBD, TCP are referred in Example 2.1. In this figure, LGL is LGL-p.

FIG. 12 shows representative cryo-TEM images of Sit: LGC Vs loading (A) 7-DHC; (B) CBD; (C) Dil/DID; (D) NCA and (E) AG.7-DHC, CBD, Dil/DID, NCA and AG are referred in Example 2.1.

FIG. 13 shows a comparison of Sit: LGL: DC-Chol active-loaded formulations in terms of particle size(S), ζ-Potential (ζ-Pot), Polydiversity Index (Pdl) and encapsulation efficiency (EE). 7-DHC, CBD, TCP, MTX, HGH. In this figure, LGL is LGL-p.

FIG. 14 shows representative cryo-TEM images of Sit: LGC Vs loading (A) 7-DHC; (B) CBD and (C) TCP. In this figure, LGL is LGL-p. 7-DHC, CBD and TCP are referred in Example 2.1.

FIG. 15 shows 7-dehydrocholesterol (7DHC) to vitamin D conversion by irradiation of 7-DHC loaded vesicles compared to free 7DHC suspensions, quantified by HPLC. “7DHC” corresponds to “7-dehydrocholesterol”, “C” corresponds to “Concentration”, “VD” corresponds to “Vitamin D, cholecalciferol”, “F” corresponds to “Free”, “V” corresponds to “loaded vesicles”, “B I” corresponds to “before irradiation”, “30′ I” corresponds to “30 min irradiation”, and “1 h I” corresponds to “1 h irradiation”.

FIG. 16 shows (A): structural differences between vesicles and emulsions (O/W and W/O). (B) cryo-TEM image in which emulsions and vesicles are seen differently due to its structural differences. “V” corresponds to “Vesicles”, “E” corresponds to “Emulsion”, “O/W” corresponds to “Oil in Water”, “W/O” corresponds to “Water in Oil”, “A” corresponds to “Aqueous”, and “O” corresponds to “Oily”.

### DETAILED DESCRIPTION OF THE INVENTION

### Definitions

Particle: The term “particle” refers to a material with at least one nano or microscopic dimension, e.g., inorganic particles, polymeric particles, tubes, gels, or lipidic particles such as solid lipid particles, liposomes, or other kind of vesicles. Particles can be referred to as carriers when they entrap molecules as a cargo that can be delivered for example to a certain tissue, or that is protected from external agents. The terms “entity” and “capsule” can be used herein similarly to “particle”.

Vesicle or vesicular system: The terms “vesicle” and “vesicular system” is used interchangeably herein, and refers to nano or microparticulate colloidal carriers, which form spontaneously when certain lipids are hydrated in aqueous media and are usually 0.02-5.0 μm in diameter. Vesicles or vesicular systems comprise an enclosed aqueous liquid compartment separated from its surroundings by one or more lipid bilayers.

Most of the self-assembling molecules possess an amphiphilic character, that is, they contain hydrophilic and hydrophobic domains in their structure. Phospholipids, surfactants, and block copolymers, which are the generally used as self-assembling monomers for the production of vesicles, are usually constituted by a long hydrophobic tail and a polar hydrophilic head group. Under aqueous conditions, this hydrophobic-hydrophilic dual character promotes their association through weak, noncovalent interactions to form ordered assemblies with different morphologies and sizes that range from nanometers to microns.

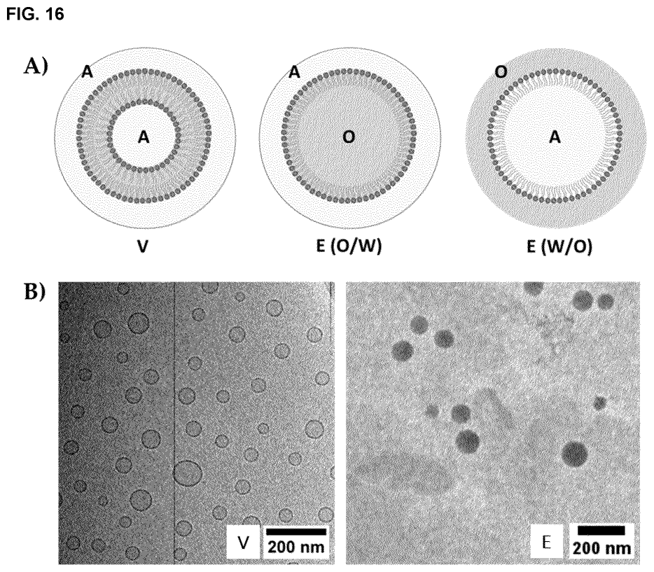

Vesicles are one type of lipid nanoparticles, but there are other types like emulsions. However, they have different structure and properties. As mentioned above, vesicles have an enclosed aqueous liquid compartment separated from its surroundings by lipid bilayers. In this sense, vesicles are surrounded by aqueous environments either from the outside and the inside. In contrast, emulsions are dispersions of oily and aqueous phases stabilized by surfactants (FIG. 16(A)). They have a stabilized core phase surrounded by the other phase (Plaza-Oliver et al. 2021). These structural differences can be also seen using microscopical techniques like cryo-TEM (FIG. 16(B)). In cryo-TEM images, “oil in water” emulsions are seen as black solid dots. They are oily droplets stabilized in an aqueous phase. On the contrary, the lipid bilayer of vesicles is observed in cryo-TEM images as the thin black circle lines, and the aqueous lumen inside vesicles show the same color than the outside environment since they are both aqueous phases.

There are other types of vesicular systems used for drug delivery including: liposomes (constituted mainly by phospholipids), vesicles containing nonionic (niosomes), cationic (cationic vesicles), or both, cationic and anionic surfactants (catanionic vesicles).

Liposome: The term “liposome” refers to a self-assembling structure comprising one or more membranes comprising lipid bilayers, each of which comprises two monolayers containing amphipathic lipid molecules oppositely oriented. Liposomes can have a single bilayer membrane (small unilamellar vesicles (SUVs) and large unilamellar vesicles (LUVs)), or multiple bilayer membrane (multilamellar large vesicles (MLVs)).

Phytosterol or plant sterol: The terms “phytosterol”, “plant sterol” or “plant-derived sterol” are used interchangeably herein and refer to a group of naturally occurring molecules found in plants, which can be classified into sterols and stanols, according to the presence or absence of a double bond in the sterol ring. Sterols in nature are biosynthetically derived from squalene and are structurally similar to cholesterol, which is an exclusively animal sterol. Sterols core structure is composed of four condensed rings: three 6-membered cyclohexane and one 5-membered cyclopentane rings, with a hydroxyl group at the 3-position of the A-ring.

Glucose-derived surfactant: The term “glucose-derived surfactant” refers to surfactants which are derived from a glucose molecule, and include alkyl polyglucosides and derivatives (e.g., methyl glucoside esters), and fatty acid glucamides. Glucose-derived surfactants are considered to have an extraordinary product safety in terms of ecological, toxicological and dermatological properties. Glucose-derived surfactants are used in detergents and dishwashing agents, among other applications.

Stabilizing agent: The term “stabilizing agent” or “stabilizer” is a component or compound which is added in a vesicular system to increase its colloidal stability.

Therapeutic agent: The term “therapeutic agent” is used herein to refer to any agent or substance capable of producing an effect on the body or area administered. As described herein, the term “therapeutic agent” can be used to refer to, e.g., a “pharmaceutical agent” or to a “cosmetic agent”.

### Vesicular Systems

One aspect of the invention relates to a vesicle comprising at least one phytosterol and at least one glucose-derived surfactant.

In some embodiments the phytosterol comprises at least one phytosterol selected from the group consisting of β-sitosterol (Sit), β-sitostanol, campesterol, campestanol, stigmasterol, brassicasterol, stigasterol, ergosterol, Δ5-avenasterol and combinations thereof. In some embodiments, the phytosterol is a combination of phytosterols. In some embodiments, the phytosterol comprises Sit. In a particular embodiment, the phytosterol comprises a combination of Sit and at least one other phytosterol. In a more particular embodiment, the combination comprises over 70% of Sit. In another embodiment, the phytosterol comprises between 70% and 95% of Sit. In a particular embodiment, the phytosterol comprises 70%, 75%, 80%, 85%, 90% or 95% of Sit.

In some embodiments, the at least one glucose-derived surfactant is selected from the group consisting of an alkyl polyglucoside surfactant (APG), a fatty acid glucamide and a methyl glucoside ester. More particularly, the glucose-derived surfactant is an APG or a derivative thereof as described herein. In a more particular embodiment, the APG or the derivative thereof has a carbon chain length of at least twelve carbons. In another embodiment, the APG or the derivative thereof has a carbon chain length of twelve, fourteen, sixteen carbons or a combination thereof. In another embodiment, the at least one glucose-derived surfactant is an APG. Particularly, the alkyl polyglucoside surfactant is lauryl glucoside (LGL).

In some embodiments, the APG is an alkyl polyglucoside derivative. Particularly, the alkyl polyglucoside derivative is selected from the group consisting of a carboxylate, a carbonate, a butyl ether, an ethoxylate, an isethionate, an ether, a sulfate, an epoxide adduct, a phosphate, a sulfosuccinate, an ester and a glycerol ether and an inorganic salt.

In another embodiment, the alkyl polyglucoside surfactant or the derivative thereof is selected from the group consisting of octyl glucoside (OGL), decyl glucoside (DGL), LGL, tetradecyl glucoside, hexadecyl glucoside, tetradecyl D-glucoside (14C), hexadecyl β-D-glucopyranoside (16C), octadecyl D-glucoside (18C), arachidyl glucoside (20C), a mixture of C12-20 alkyl glucoside, cetearyl glucoside (a mixture of C18-20 alkyl glucoside), C20-22 alkyl glucoside, coco-glucoside (alkyl chain residue of fatty alcohols derived from coconut acid), isostearyl glucoside (branched), ocyldodecyl glucoside (branched), phosphorus derivatives of alkyl polyglucoside and lauryl glucoside carboxylate (LGC).

In some embodiments, the APG is selected from the group consisting of LGL, tetradecyl glucoside, hexadecyl glucoside or a combination thereof. In a particular embodiment, the APG further comprises OGL and/or DGL. In a particular embodiment, the APG is LGL. In another embodiment, the APG is LGL in combination with one or more other APGs. In a particular embodiment, the APG comprises a combination of LGL, tetradecyl glucoside and hexadecyl glucoside. In some embodiments, the APG comprises a combination of OGL, DGL, LGL, tetradecyl glucoside and/or hexadecyl glucoside.

In some embodiments, the glucose-derived surfactant comprises a fatty acid glucamide. Particularly, the glucamide is selected from the group consisting of lauroyl methyl glucamide, myristoyl methyl glucamide, cocoyl methyl glucamide, sunfloweroil methyl glucamide, glucamine oxides and betaines, anionic glucamides, bifunctional glucamides and combinations thereof. In some embodiments, the glucose-derived surfactant comprises a fatty acid glucamide in combination with at least one other glucose-derived surfactant.

In some embodiments, the vesicle further comprises at least one additive. In a particular embodiment, the additive is a surfactant. More particularly, the vesicle further comprises at least surfactant selected from the group consisting of sodium lauroyl sarcosinate (SLS), sodium laurate (SL), sodium dodecyl sulfate (SDS), sodium lauroyl glycinate, hydrochloride salt of N3-lauroyl lysine methyl ester and combinations thereof. In some embodiments, the vesicle comprises a glucose-derived surfactant and further comprises at least one compound selected from the group consisting of LGC, SLS, SL, SDS, sodium lauroyl glycinate, hydrochloride salt of N3-lauroyl lysine methyl ester and combinations thereof. In a particular embodiment, the glucose-derived surfactant comprises at least one APG. More particularly, the APG is LGL and the vesicle further comprises at least one compound selected from the group consisting of LGC, SLS, SL, SDS, sodium lauroyl glycinate, hydrochloride salt of N3-lauroyl lysine methyl ester and combinations thereof.

As shown in the Examples of the present invention, the glucose-derived surfactant (e.g., APG or LGL) can be substituted, totally or partially, by other compounds such as other APGs (e.g., LGC) or other compounds considered additives (e.g., SL or SLS). Therefore, in some embodiments, the glucose-derived surfactant (e.g., APG) is totally or partially substituted by another compound. In some embodiments, the glucose-derived surfactant (e.g., APG) is partially substituted by a compound selected from the group consisting of LGC, SLS, SL, SDS, sodium lauroyl glycinate, hydrochloride salt of N3-lauroyl lysine methyl ester and combinations thereof. In a particular embodiment, the glucose-derived surfactant is an APG and is partially substituted by another compound. In a more particular embodiment, the APG is LGL and is partially substituted by at least one compound selected from the group consisting of LGC, SL and SLS.

In another embodiment, the glucose-derived surfactant (e.g., APG) is totally substituted by another compound. In some embodiments, the glucose-derived surfactant (e.g., APG) is totally substituted by at least one compound selected from the group consisting of LGC, SLS, SL, SDS, sodium lauroyl glycinate, hydrochloride salt of N3-lauroyl lysine methyl ester and combinations thereof. In a particular embodiment, the glucose-derived surfactant (e.g., APG) is totally substituted by LGC. In a more particular embodiment, the glucose-derived surfactant is an APG, and the APG is totally substituted by LGC. More particularly, the APG is LGL and the APG is totally substituted by LGC.

Alternatively, the invention relates to a vesicle comprising a phytosterol and at least one glucose-derived surfactant, wherein the at least one glucose-derived surfactant is an APG selected from LGL and/or LGC. In some embodiments, the APG is a combination of LGL and LGC. In some embodiments, the APG is LGL. In another embodiment, the APG is LGC. In some embodiments, the vesicle further comprises an additive selected from SL and/or SLS.

In another embodiment, the vesicle comprises at least one phytosterol and at least one glucose-derived surfactant, wherein the surfactant has a purity selected from at least 80%, 90%, 95%, 96%, 97%, 98% and 99%. More particularly, the surfactant has a purity of at least 98%. In another embodiment, the vesicle comprises at least one phytosterol and an APG. In some embodiments, the APG is LGL. Particularly, the LGL has a purity selected from at least 80%, 90%, 95%, 96%, 97%, 98% and 99%. More particularly, the LGL has a purity of at least 98%. In a particular embodiment, the at least one phytosterol comprises Sit.

In another embodiment, the vesicle comprises at least one phytosterol and at least one glucose-derived surfactant, wherein the molar ratio phytosterol: glucose-derived surfactant is between 0.05-1:0.2-2. In a particular embodiment, the molar ratio phytosterol: glucose-derived surfactant is between 0.2-1:0.2-2. Particularly, the molar ratio phytosterol: glucose-derived surfactant is 1:0.75-2. More particularly, the molar ratio phytosterol: glucose-derived surfactant is 1:1. In another embodiment, the glucose-derived surfactant is an APG and the molar ratio phytosterol: APG is between 0.05-1:0.2-2 and particularly between 0.2-1:0.2-2. In a particular embodiment, the molar ratio phytosterol: APG is 1:0.75-2 and more particularly, 1:1. In some embodiments, the APG is LGL. In other embodiments, the phytosterol comprises Sit.

In some embodiments, the vesicle is for use in at least one composition selected from the group consisting of pharmaceutical composition, in a cosmetic composition, in a nutraceutical composition (e.g., a food supplement), in a food composition, in a food additive and in a detecting and diagnostic composition.

Stabilizing agents: Neutral additives

Vesicles (Vs) are modified to increase their colloidal stability through the addition of molecules capable of increasing such stability without compromising other desirable properties of the Vs. Molecules used to increase such stability are considered stabilizing agents.

In some embodiments, the vesicle further comprises a stabilizing agent. In an embodiment, the stabilizing agent is selected from the group consisting of a neutral additive, a cationic additive, and an anionic additive.

In particular embodiment, the neutral additive is selected from the group consisting of a polyethylene glycol (PEG) moiety, a PEGylated fatty acid, a PEGylated phospholipid, a polysaccharide, a zwitter-ionic polypeptide, a poly amino acid-based polymer, a poly(2-oxazoline)-based polymer, polyvinyl pyrrolidine, other PEGylated membrane components and a non-ionic surfactant. In a particular embodiment, the polysaccharide is chitosan. In a particular embodiment, the poly amino acid-based polymer is selected from the group consisting of polyglutamic acid, poly(hydroxyethyl-L-asparagine and poly(hydroxyl ethyl-L-glutamine). In a particular embodiment, the PEGylated membrane component is a PEGylated aliphatic amine. In a particular embodiment, the non-ionic surfactant is Tween 80.

In a particular embodiment, the neutral additive is a PEGylated fatty acid. Particularly, the PEGylated fatty acid is selected from the group consisting of PEGylated cholesterol, dipalmitoyl phosphatidyl-choline (DPPC), distearoyl phosphatidylethanolamine-polyethylene glycol 2000 (DSPE-PEG2000). In a more particular embodiment, the PEGylated fatty acid is PEGylated cholesterol.

PEGylation is the process through which molecules and macrostructures are modified by their conjugation with a PEG molecule or its derivatives, which are non-toxic and non-immunogenic polymers. PEG and its derivatives which can be used for such modification are considered PEGylating agents. In other embodiments, the PEGylating agent is selected from the group consisting of PEG, PEG monostearate, PEG stearate and PEG stearate 4. In a particular embodiment, the PEGylating agent is PEG.

PEGylated cholesterol is a conjugate comprising a cholesterol moiety (Chol) and a PEG moiety. In a particular embodiment, the cholesterol moiety and the PEG moiety are covalently attached. Particularly, the conjugate has a structure Chol-PEGn-X, wherein n is the number of PEG monomers of the PEG moiety. More particularly, n is selected from 50 to 2000. In some embodiments, n is selected from 200 to 1000. Particularly, n is selected from the group consisting of 200, 300, 400, 500, 600, 700, 800, 900 and 1000. More particularly, n is 600. In another embodiment, X is selected from the group consisting of —SH, —OH, —CHO, —OCH₃, —NH₂, —NH, —CH₃, —N₃, —COOH,-maleimide, a peptide, an antibody and a sugar.

In some embodiments, the vesicle comprises at least one phytosterol and at least one glucose-derived surfactant, further comprising a neutral additive. Particularly, the neutral additive is a PEGylated fatty acid. In some embodiments, the percentage of the neutral additive in respect to the phytosterol is between 1-20%. In another embodiment, the percentage of the neutral additive in respect to the phytosterol is selected from the group consisting of 1%, 2%, 3%, 4%, 5%, 6%, 7%, 8%, 9%, 10%, 11%, 12%, 13%, 14%, 15%, 16%, 17%, 18%, 19% and 20%. In a particular embodiment, the percentage of the neutral additive in respect to the phytosterol is between 5-15%. In a more particular embodiment, the percentage of the neutral additive in respect to the phytosterol is between 6-10%. In a more particular embodiment, the percentage of the neutral additive in respect to the phytosterol is 7%. In another embodiment, the ratio phytosterol: glucose-derived surfactant: neutral additive is 0.90-0.99:1:0.01-0.1. In a particular embodiment, the ratio phytosterol: glucose-derived surfactant: neutral additive is selected from the group consisting of 0.90:1:0.1, 0.91:1:0.09, 0.92:1:0.08, 0.93:1:0.07 and 0.94:1:0.06. In a particular embodiment, the neutral additive is a PEGylated fatty acid, particularly PEGylated cholesterol. In another embodiment, the phytosterol comprises Sit. In another embodiment, the glucose-derived surfactant is an APG.

### Stabilizing Agents: Charged Additives

Vesicles can be modified to increase their stability through the addition of charged molecules, i.e., charged additives capable of increasing such stability without compromising other desirable properties of the Vs. Therefore, charged additives are a type of stabilizing agents used in the present invention. The addition of such charged molecules is used to modify the electrical charge of the Vs.

Vesicles which comprise positively charged additives, i.e., cationic additives, are ultimately positively charged. Contrarily, Vs which comprise negatively charged additives, i.e., anionic additives, are ultimately negatively charged. The modification of the Vs electrical charge by the addition of charged additives results in a higher encapsulation efficiency of certain molecules, which have a higher encapsulation capacity in electrically charged Vs, whether positive or negative, rather than electrically neutral vesicles. In some embodiments, Vs which further comprise an anionic additive and are consequently negatively charged, are useful for the encapsulation of positively charged molecules. Similarly, Vs which further comprise a cationic additive and are consequently positively charged, are useful for the encapsulation of negatively charged molecules.

Therefore, in some embodiments, the vesicle further comprises a stabilizing agent, particularly a charged additive. In a particular embodiment, the charged additive is selected from an anionic additive and a cationic additive. In another embodiment, the additive is located in the membrane of the vesicle. Alternatively, the additive is located inside the vesicle. In some embodiments, the additive is coupled to the membrane of the vesicle. In another embodiment, the additive is interacting by surface charges with the vesicle.

In a particular embodiment, the additive is an anionic additive (i.e., negatively charged). In some embodiments, the vesicle further comprises an anionic additive and the vesicle is used in a cosmetic composition. In another embodiment, the anionic additive is located in the membrane of the vesicle. Alternatively, the anionic additive is located inside the vesicle. In a particular embodiment, the anionic additive is selected from the group consisting of LGC, SLS, SL, SDS, di-sodium glucoside citrate, sodium glucoside tartrate, di-sodium glucoside sulfosuccinate, an amino acid-derived surfactant, an anionic phospholipid, an anionic lipid, and combinations thereof. In a particular embodiment, the anionic additive is selected from the group consisting of LGC, SLS, SL, and combinations thereof. In another particular embodiment, the anionic phospholipid is 1,2-dioleoyl-sn-glycero-3-phospho-L-serine (DOPS). In another particular embodiment, the anionic lipid is selected from cholesterol sulfate, cholesterol phosphate and cholesteryl acetate.

In another embodiment, the ratio phytosterol: glucose-derived surfactant: anionic additive is between 1:1:0.05-0.1. In a particular embodiment, the ratio phytosterol: glucose-derived surfactant: anionic additive is 1:1:0.05, 1:1:0.08 or 1:1:0.1. In a particular embodiment, the anionic additive is selected from the group consisting of LGC, SLS, SL, and a combination thereof, and the ratio phytosterol: glucose-derived surfactant: anionic additive is between 1:1:0.05-0.1. In a particular embodiment, the anionic additive is LGC, and the ratio phytosterol: glucose-derived surfactant: anionic additive is 1:1:0.05. In another embodiment, the anionic additive is SLS, and the ratio phytosterol: glucose-derived surfactant: anionic additive is 1:1:0.08. In another embodiment, the anionic additive is SL, and the ratio phytosterol: glucose-derived surfactant: anionic additive is 1:1:0.1. In some embodiments the phytosterol comprises Sit. In some embodiments, the glucose-derived surfactant is an APG, particularly LGL.

In another embodiment, the ratio phytosterol: glucose-derived surfactant: anionic additive is between 1:0-0.8:0.2-1. In a particular embodiment, the ratio phytosterol: glucose-derived surfactant: anionic additive is selected from the group consisting of 1:0:1, 1:0.2:0.8, 1:0.5:0.5 and 1:0.8:0.2. In a more particular embodiment, the anionic additive is LGC and the ratio phytosterol: glucose-derived surfactant: anionic additive is between 1:0-0.8:0.2-1. In some embodiments, the phytosterol comprises Sit. In some embodiments, the glucose-derived surfactant is an APG, particularly LGL.

In some embodiments, the additive is a cationic additive which is positively charged. In a particular embodiment, the vesicle further comprises a cationic additive and the vesicle is used in a pharmaceutical composition. In another embodiment, the cationic additive is located inside the vesicle. Alternatively, the anionic additive is located in the membrane of the vesicle. In a particular embodiment the cationic additive is selected from the group consisting of a cationic lipid, a cationic phospholipid, a surfactant comprising quaternary amines, and an amino acid-derived surfactant. In a particular embodiment, the cationic lipid is DC-cholesterol (3α-[N-(N′, N′-dimethylaminoethane)-carbamoyl]cholesterol hydrochloride, DC-Chol). In another particular embodiment, the surfactant comprising quaternary amines is selected from the group consisting of stearalkonium chloride, dicetyldimonium chloride, behentrimonium chloride and cetrimide.

In a particular embodiment, the ratio phytosterol: glucose-derived surfactant: cationic additive is between 0-1:1:0.05-1. In a more particular embodiment, the ratio phytosterol: glucose-derived surfactant: cationic additive is between 0-0.8:1:0.2-1. In another particular embodiment, the ratio phytosterol: glucose-derived surfactant: cationic additive is selected from the group consisting of 1:1:0.5, 0.8:1:2, 0.5:1:0.5, 0.2:1:0.8 and 0:1:1. Particularly, the cationic additive is DC-Chol. In some embodiments, the phytosterol comprises Sit. In some embodiments, the glucose-derived surfactant is an APG, particularly LGL.

### Shape and Size

In some embodiments, the vesicle has a circular shape or similar. Alternatively, in some embodiments, the vesicle has an oval shape or similar.

In some embodiments, the vesicle has an average diameter of at least 20 nm. In another embodiment, the vesicle has an average diameter of at least 50 nm. In another embodiment, the vesicle has an average diameter between 20 nm and 5 μm. In a particular embodiment, the vesicle has an average diameter between 50 and 800 nm. In a particular embodiment, the average diameter is selected from the group consisting of 50-200 nm, 100-300 nm and 200-800 nm. In another embodiment, the vesicle is positively charged and its average diameter is 50-200 nm. In another embodiment, the vesicle is negatively charged and its average diameter is 100-300 nm. In another embodiment, the vesicle is neutral and its average diameter is 200-800 nm.

### Encapsulation of Agents

In some embodiments, the vesicle is capable of encapsulating an agent, also referred herein as active ingredient. In one embodiment, the vesicle is capable of encapsulating at least one agent selected from the group consisting of a pharmaceutical agent, a cosmetic agent, a food additive, a vitamin, a diagnosing agent and a detecting agent. In some embodiments, the vesicle is capable of encapsulating at least one agent selected from the group consisting of a small chemical molecule, a biomolecule and a conjugate thereof. In a particular embodiment, the biomolecule is selected from the group consisting of a peptide, a hormone, a metabolite, an antibody, a protein, an enzyme, an oligonucleotide, a nucleic acid, a carbohydrate and a lipid. In a particular embodiment, the agent is encapsulated inside the vesicle. In another embodiment, the agent is encapsulated in the membrane of the vesicle. In some embodiments, the agent is coupled to the membrane of the vesicle. In another embodiment, the agent is interacting by surface charges with the vesicle. In another embodiment, the vesicle further comprises an agent. Particularly, the agent is selected from the group consisting of a pharmaceutical agent, a cosmetic agent, a food additive, a vitamin, a diagnosing agent and a detecting agent.

In some embodiments, the vesicle further comprises a pharmaceutical agent. In a particular embodiment, the pharmaceutical agent is lipophilic. In another embodiment, the pharmaceutical agent is hydrophilic. In another particular embodiment, the pharmaceutical agent is hydrophilic. In another embodiment, the pharmaceutical agent is selected from the group consisting of a small molecule, a peptide, a hormone, a metabolite, an antibody, a protein, an enzyme, an oligonucleotide, a nucleic acid, a carbohydrate, a lipid and a conjugate thereof.

In some embodiments, the vesicle further comprises a cosmetic agent. In a particular embodiment, the cosmetic agent is lipophilic. In another embodiment, the cosmetic agent is hydrophilic. In another embodiment, the cosmetic agent is selected from the group consisting of a small molecule, a peptide, a hormone, a metabolite, an antibody, a protein, an enzyme, an oligonucleotide, a nucleic acid, a carbohydrate, a lipid and a conjugate thereof. In a particular embodiment, the cosmetic agent is a small molecule. In another embodiment, the cosmetic agent is a peptide.

In some embodiments, the vesicle further comprises a food additive. In a particular embodiment the food additive is selected from the group consisting of a small chemical molecule, a biomolecule and a conjugate thereof. In a particular embodiment, the biomolecule is selected from the group consisting of a peptide, a hormone, a metabolite, an antibody, a protein, an enzyme, an oligonucleotide, a nucleic acid, a carbohydrate and a lipid. In another embodiment the food additive is selected from a vitamin and a mineral.

In some embodiments, the vesicle further comprises a diagnosing or detecting agent. The diagnosing or detecting agent can also be referred to as tracking agent or labelling agent. These agents can be used to label the vesicle (e.g., using a fluorescent dye as a tracking/labelling agent) to track the Vs distribution, and optionally track the delivery of agents/active ingredients (e.g., pharmaceutical or cosmetic agents of interest). In a particular embodiment, the diagnosing or detecting agent is selected from the group consisting of a dye, a reagent and a biomarker. More particularly, the diagnosing or detecting agent is a dye, particularly a fluorescent dye. In some embodiments, the vesicle is capable of detecting a specific molecule. In a particular embodiment, said molecule is selected from the group consisting of a peptide, a hormone, a metabolite, an antibody, a protein, an enzyme, an oligonucleotide, a nucleic acid, a carbohydrate and a lipid.

In another embodiment, the vesicle comprises a diagnosing or detecting agent and the vesicle is used as a bioimaging tool to track the delivery of an agent. In another embodiment, the vesicle comprises a labelling agent, a targeting ligand and a therapeutic agent. Particularly, the vesicle comprises a dye for labelling, a targeting ligand for site-specific labelling and a therapeutic agent to be delivered. Thus, in some embodiments, the invention relates to the use of a vesicle as described herein as a bioimaging tool.

In some embodiments, the vesicle is capable of encapsulating at least one lipophilic agent. In some embodiments, the lipophilic agent is a lipophilic small molecule. In a particular embodiment, the lipophilic small molecule is selected from the group consisting of 7-dehydrocholesterol, cannabidiol (CBD), α-Tocopherol (TCP) and carbocyanine dyes. In some embodiments, the vesicle is capable of encapsulating at least one hydrophilic agent. In some embodiments, the hydrophilic agent is a hydrophilic vitamin. In a particular embodiment, the hydrophilic small molecule is selected from the group consisting of niacinamide or vitamin B3 and ascorbyl glucoside (AG). In some embodiments, the vesicle is capable of encapsulating at least one small protein. In a particular embodiment, the small protein is basic Fibroblast Growth Factor (bFGF).

### Compositions

In another aspect, the invention also relates to a composition comprising a plurality of vesicles as defined herein, i.e., vesicles (Vs) which comprise at least one phytosterol (e.g., β-sitosterol (Sit) and at least one glucose-derived-surfactant. Particularly, the Vs comprise Sit and at least one alkyl polyglucoside surfactant (APG).

In some embodiments, the composition is selected from the group consisting of a pharmaceutical composition, a cosmetic composition, a nutraceutical composition (e.g., a food supplement), a food composition, a food additive and a detecting or diagnostic composition.

Thus, another aspect of the invention relates to a pharmaceutical composition comprising a therapeutically effective amount of Vs as defined herein and at least one pharmaceutically acceptable excipient, vehicle or carrier. In some embodiments, the at least one acceptable excipient, vehicle or carrier is selected from the group consisting of hydroxypropylmethylcellulose, polymethacrylate-based copolymers, polyethyleneglycol, polyvinylpyrrolidone, polyvinyl methyl ether/maleic acid, ethanol, isopropyl alcohol, linolin, alginate, starch, hyaluronic acid, cellulose, water, wax, agar, pectin, sucrose, maltose, lactose, trehalose, and inorganic salts.

In another aspect, the invention relates to a cosmetic composition comprising a cosmetically effective amount of Vs as defined herein and at least one cosmetically acceptable excipient, vehicle or carrier. In some embodiments, the at least one acceptable excipient, vehicle or carrier is selected from the group consisting of hydroxypropylmethylcellulose, carbomer, alginate, water, myristyl myristate, paraffin, glycerin, mineral oil, polyethylene glycol, butylene glycol, xanthan gum, lanolin, cetyl alcohol, cetearyl alcohol, sodium benzoate, potassium sorbate, phenoxyethanol, benzyl alcohol, trehalose and sucrose.

In some embodiments, the cosmetic composition is formulated in the form of a hydrogel. Particularly, the composition is formulated in the form of a hydrogel and comprises at least one acceptable excipient, vehicle or carrier selected from the group consisting of hydroxypropylmethylcellulose, carbomer and alginate. In another embodiment, the cosmetic composition is formulated in the form of an emulsion. Particularly, the composition formulated in the form of an emulsion comprises at least one acceptable excipient, vehicle or carrier selected from the group consisting of water, myristyl myristate, paraffin, glycerin, mineral oil, polyethylene glycol, butylene glycol, xanthan gum, lanolin, cetyl alcohol and cetearyl alcohol. In another embodiment, the cosmetic composition is formulated in the form of a semisolid composition. Particularly, the composition formulated in the form of a semisolid composition comprises at least one acceptable excipient, vehicle or carrier selected from the group consisting of sodium benzoate, potassium sorbate, phenoxyethanol and benzyl alcohol. In some embodiments, the cosmetic composition is formulated in the form of a liquid composition. Particularly, the composition formulated in the form of a liquid composition comprises at least one acceptable excipient, vehicle or carrier selected from the group consisting of sodium benzoate, potassium sorbate, phenoxyethanol and benzyl alcohol. These four compounds (i.e., sodium benzoate, potassium sorbate, phenoxyethanol and benzyl alcohol) are used as preservative for semisolid and liquid compositions. In some embodiments, the cosmetic composition is formulated in the form of a solid composition. Particularly, the composition formulated in the form of a solid composition comprises at least one acceptable excipient, vehicle or carrier selected from trehalose and sucrose.

In another aspect, the invention relates to a nutraceutical composition (e.g., food supplement) comprising a nutraceutically effective amount of Vs as defined herein and at least one nutraceutically acceptable excipient, vehicle or carrier. In some embodiments, the at least one acceptable excipient, vehicle or carrier is selected from the group consisting of trehalose, sucrose, mono- and diglycerides of fatty acids, starch and maize-based polymers, alginate and gum base.

In another aspect, the invention relates to a food composition/food additive comprising an effective amount of Vs as defined herein and at least an acceptable excipient, vehicle or carrier. In some embodiments, the at least one acceptable excipient, vehicle or carrier is selected from the group consisting of trehalose, sucrose, mono- and diglycerides of fatty acids, starch and maize-based polymers, alginate and gum base.

In another aspect, the invention relates to a detecting or diagnostic composition comprising an effective amount of Vs as defined herein and at least an acceptable excipient, vehicle or carrier. In some embodiments, the at least one acceptable excipient, vehicle or carrier is a dye, particularly, a fluorescent dye.

In some embodiments, the composition is administered topically, orally, intravenously, through inhalation, parenterally, through mucosal administration, subcutaneously or ocularly. Particularly, the composition is administered topically.

### Applications of the Vesicles/Compositions

In another aspect, vesicles (Vs) or a composition comprising the Vs as described herein are used in the manufacture of a suspension. Alternatively, this aspect relates to the use of the Vs or the composition as described herein for the manufacture of a suspension. In some embodiments the suspension is for topical, oral, intravenous, inhalation, parenteral, mucosal, subcutaneous or ocular administration.

In another aspect, the Vs or the composition thereof as described herein is used as a delivery system. Alternatively, this aspect relates to the use of the Vs or the composition as described herein as a delivery system. In a particular embodiment, the composition is used as a delivery system of at least one active ingredient. In some embodiments, the active ingredient is selected from the group consisting of a pharmaceutical agent, a cosmetic agent, a food additive, a vitamin, a diagnosing agent and a detecting agent. More particularly, the active ingredient is a pharmaceutical or a cosmetic agent. In some embodiments, pharmaceutical or cosmetic agents.

Another aspect relates to the Vs or the composition as defined herein for use as a medicament. In another aspect, the Vs or the composition as described herein is for use in the treatment, diagnostic or prevention of a disease. This aspect can be alternatively formulated as a method of treatment, diagnostic or prevention of a disease comprising administering to a subject in need thereof. Vs or a composition as defined herein.

In some embodiments, the Vs or the composition as described herein is used as a diagnostic or detection tool, particularly a bioimaging tool. Alternatively, this aspect relates to the use of the Vs or the composition as described herein as a diagnostic or detection tool, particularly a bioimaging tool.

Particularly, the composition comprises a diagnostic or detecting agent (herein also referred to as tracking agent or labelling agent). More particularly, the composition comprises a diagnostic or detecting agent, a targeting ligand and an active ingredient to be delivered. Alternatively, the composition comprises a diagnostic or detecting agent, a targeting ligand and a therapeutic agent to be delivered.

In some embodiments, the Vs or the compositions as described herein are capable of penetrating cells or detecting specific cell types, tissues or molecules. Therefore, in some embodiments, the composition further comprises cell-penetrating or targeting moieties. In a particular embodiment, the moiety is a peptide.

In some embodiments, the cell-penetrating or targeting moiety is selected from the group consisting of Arginylglycylaspartic acid (RGD), Palmitoyl Tripeptide-1 (Pal-GHK), melanostatin DM (HRAWFK), AHK Copper (AHK2Cu) and transferrin. In some embodiments, the Vs or the compositions comprisesing Vs are capable of contributing to cell attachment, cell spreading, actin-skeleton formation and/or focal-adhesion formation, stimulating the release of specific proteins, are capable of crossing the blood-brain barrier and/or have an anti-aging effect. In a particular embodiment, the Vs or the compositions comprising Vs further comprise RGD and are capable of contributing to cell attachment, cell spreading, actin-skeleton formation and/or focal-adhesion formation. In a particular embodiment, the Vs or the compositions comprising Vs further comprise Pal-GHK and are capable of stimulating the release of collagen and decorin. In a particular embodiment, the Vs or the compositions comprisesing Vs further comprise transferrin and are capable of crossing the blood-brain barrier. In a particular embodiment, the Vs or the compositions comprising Vs further comprise HRAWFK and/or AHK2Cu and have an anti-aging effect.

### Methods

Another aspect of the invention relates to a method to produce vesicles, as described above, using the DELOS-susp methodology. CO₂-based DELOS-SUSP methodology ensures robustness and the reproducible scale up of Vs production.

In some embodiments, the invention relates to a method to produce vesicles, using the DELOS-susp methodology, which comprises:
a) preparing an organic solution comprising the components of the vesicle to be formed, i.e., at least one phytosterol, and optionally at least one glucose-derived surfactant and/or stabilizing agents, solubilized in an organic solvent e.g., ethanol, and optionally lipophilic actives, and loading said solution at atmospheric pressure in a vessel;
b) adding the liquid compressed CO₂ in the vessel and forming an CO₂-expanded solution including all components dissolved, at a CO₂ molar fraction of X CO₂ between 0.1 and 0.7, working temperature between 3° and 60° C. and working pressure of between 85 and 115 bar; and
c) depressurizing the CO₂-expanded solution into an aqueous solution optionally comprising at least one glucose-derived surfactant and/or hydrophilic actives, by adding a flow of N₂ at a working pressure of between 85 and 135 bar and maintaining a constant pressure inside the vessel.

Thus, the glucose-derived surfactant can be solubilized in an organic solution or in an aqueous solution.

In an embodiment, the membrane components of the organic solution of step (a) comprise phytosterols, glucose-derived surfactants and/or stabilizing agents. In a particular embodiment, the stabilizing agents are selected from neutral additives and charged additives. More particularly, the stabilizing agents are selected from the group consisting of PEGylated fatty acids, cationic additives, anionic additives or a combination thereof. In some embodiments, the stabilizing agents are selected from the group consisting of Chol-PEG, DC-Chol, SLS, SL and a combination thereof. In some embodiments, the phytosterol comprises Sit. In another embodiment, the glucose-derived surfactant is an APG, particularly LGL.

Surfactants can be added within the organic solution added in step (a), when the CO₂ is added in step (b) or in the aqueous solution used in step (c). Therefore, in some embodiments, the organic solution of step (a) comprises at least one glucose-derived surfactant. In another embodiments, step (b) of the method further comprises adding at least one glucose-derived surfactant in the vessel. Further, in some embodiments, the aqueous solution of step (c) comprises water and/or buffer. Particularly, the aqueous solution of step (c) further comprises at least one glucose-derived surfactant. In some embodiments, the at least one glucose-derived surfactant is an APG, particularly LGL.

In some embodiments, the CO₂ molar fraction (X CO₂) is between 0.1 and 0.7. In a particular embodiment, the CO₂ molar fraction (X CO₂) is between 0.3 and 0.5. More particularly, the CO₂ molar fraction (X CO₂) is 0.3 or 0.5.

In some embodiments, the working temperature is between 30° C. and 60° C. In a particular embodiment, the working temperature is 40° C. In another embodiment, the working temperature is 60° C. In some embodiments, the method comprises increasing the temperature of step (c) to 60° C.

In some embodiments, the flow of N₂ is added at a working pressure of between 85 and 135 bar. Particularly, the flow of N₂ is added at a working pressure of between 100 and 135 bar.

In some embodiments, the invention relates to a method to produce vesicles, using the Thin Film Hydration (TFH) methodology, which comprises:
a) preparing an organic solution comprising the components of the vesicle to be formed, i.e., at least one phytosterol, at least one glucose-derived surfactant, and/or optionally stabilizing agents, solubilized in an organic solvent, e.g., chloroform, and optionally lipophilic actives;
b) solvent evaporation forming a film with the lipophilic membrane components; and
c) hydration of the film using an aqueous solution optionally comprising hydrophilic actives.

### EXAMPLES

### Example 1: Development of a new carrier platform

The inventors of the present invention have developed a platform of homogenous Vs prepared using plant-derived ingredients and sugar-based surfactants (i.e., glucose-derived surfactants). Different vesicular systems have been prepared applying the DELOS-susp methodology and fully characterized in terms of size, stability and morphology.

### 1.1 Materials and methods

### Materials

Phytopin DERMexpert, a mixture of pine phytosterols containing 79.7% of β-sitosterol (Sit), was obtained from Purextract. Plantacare® 1200 UP (LGL-mC₁₂₋₁₆), Plantacare® 2000 UP (DGL-mC₈₋₁₆), Plantacare® 810 UP (DGL-mC8-10) and Plantapon® LGC Sorb (LGC) were provided by BASF. Dodecyl β-D-glucopyranoside (LGL-p) and decyl β-Dglucopyranoside (DGL-p) were obtained from Car-bosynth Ltd. Sodium laurate (SL) was provided by TCI Europe N.V. 3α-[N-(N′,N′-dimethylami-noethane)-carbamoyl]cholesterol hydrochloride (DC-Chol) and Cholesterol-polyethylene glycol-600 (Chol-PEG600) were purchased from Merck. Ethanol HPLC grade was purchased from Scharlab. Carbon dioxide and nitrogen were supplied by Carburos Metálicos S.A. The water used was pre-treated with a MilliQ Advantage A10 water purification system (Millipore). N-Octyl-beta-D-glucopyranoside (OGL-p) and N-Lauroylsarcosine sodium salt (SLS) were purchased from AlfaAesar. Decyl β-D-glucopyranoside was purchased from Carbobsynth.

### Production Methods of Vesicular Systems

### i) DELOS-Susp Equipment Configuration

The configuration consists of a 6-50 mL high pressure vessel, for which the temperature is maintained using an external fluid heating jacket and whose temperature and pressure are controlled by a temperature controller and a pressure indicator controller. CO₂ is pumped into the reactor through a thermostatic syringe pump (model 260D, ISCO Inc., Lincoln, US) to introduce CO₂ inside the vessel through two valves until reaching working pressure. A variable speed stirrer ensures the homogeneity of the mixture in the volumetrically expanded phase. Further, by using a depressurization micrometric valve, the expanded liquid solution contained in the vessel is depressurized into an aqueous phase placed in a collector at atmospheric pressure, while at the same time, pressure of nitrogen is adjusted by a Pressure Adjustment Valve and introduced through two valves directly from a pressurized reservoir to the vessel.

### ii) Preparation of Vesicles: Depressurization of an Expanded-Liquid Organic Solution (DELOS-Susp)

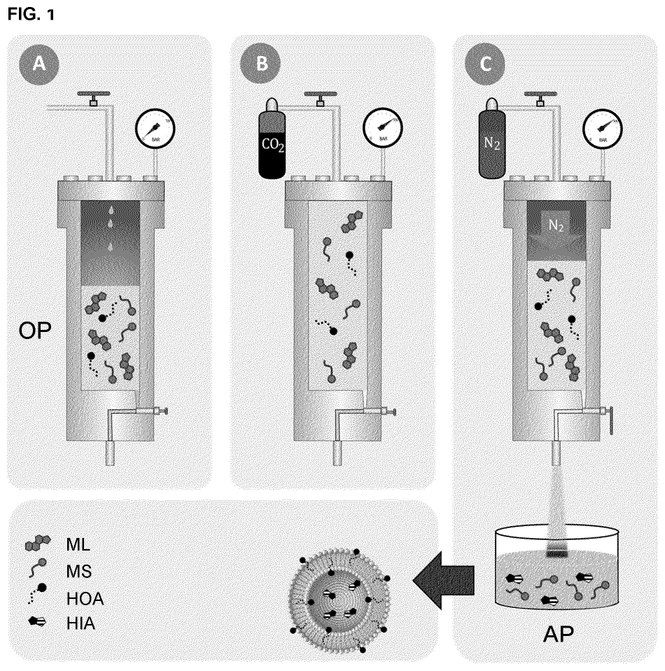

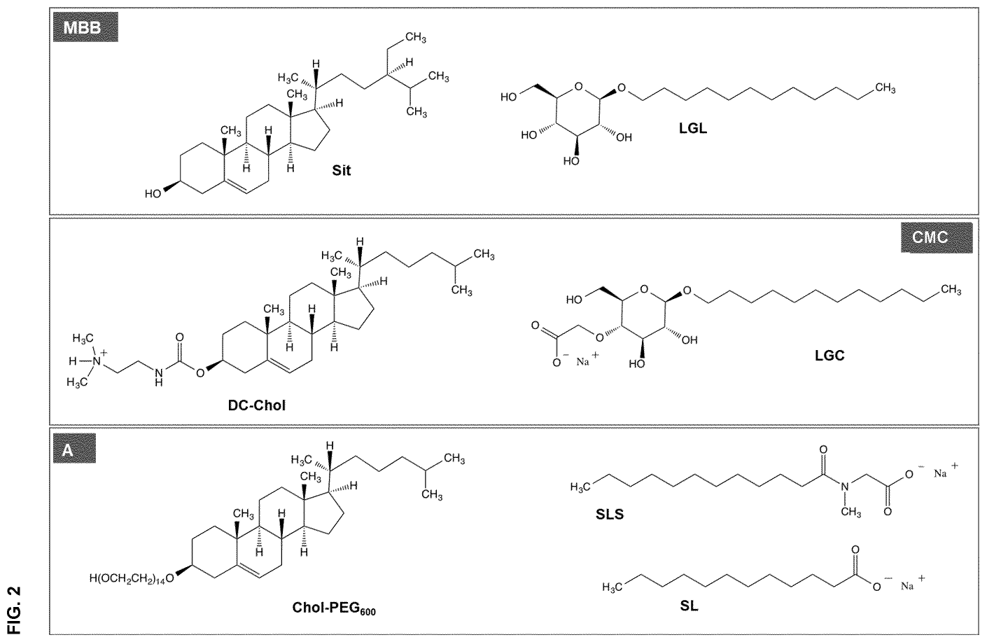

As shown in FIG. 1, the procedure includes: (a) Loading of the organic solution containing the membrane components solubilized in ethanol (e.g., β-sitosterol, LGL-p, DGL-p, OGL-p, Chol-PEG, DC-Chol), SLS and/or SL) at atmospheric pressure in the vessel; (b) Addition of liquid compressed CO₂ and formation of a CO₂-expanded solution with all the membrane components dissolved, at a CO₂ molar fraction of XCO₂=0.3-0.5, Tw=40° C. and working pressure, Pw=85-115 bar; (c) Depressurization of the CO₂-expanded solution into an aqueous solution containing the water soluble surfactants (mixture of glucose-derived surfactants (i.e., APGs) at the desired concentration. A flow of Nitrogen (N₂) at a working pressure of Pw=100-135 bar is used to plunge the CO₂-expanded solution from the reactor, maintaining a constant pressure inside the vessel during depressurization. Average time per experiment was 30 min. All the samples were filtered with 0.45 μm pore size polyethersulfone (PES) membrane syringe filters to eliminate impurities coming from the starting materials (mainly Sit and APGs mixtures). Consequently, homogeneous and opalescent colloidal dispersions of Vs in water with 15% of ethanol (v/v) were obtained. Vesicles (Vs) were stored at 4° C. until further characterization. Surfactants can be added within the organic solution added in step (a), when the CO₂ is added in step (b) or in the aqueous solution formed during step (c). The molecular structures of sterols and surfactants used herein are shown in FIG. 2.

iii) Preparation of Vesicles by Thin Film Hydration (TFH)

TFH procedures included: (a) Dissolution of the lipophilic membrane components in chloroform (e.g., β-sitosterol, LGL-p, DGL-p and/or OGL-p); (b) evaporation of the organic solvent forming a film composed of the membrane components using a rotary evaporator; and (c) hydration of the film with 10 mL of an aqueous solution at 60° C. with magnetic agitation for 30 minutes. Finally, after one day of production, the sample was sonicated at 60° C. for 30 minutes.

### Characterization of the Vesicles

### i) Size, Polydispersity Index and ζ-Potential Characterization

Mean particle size, particle size distribution (or polydispersity index, Pdl) and apparent ζ-potential of all the vesicles produced were measured using a dynamic light scattering (DLS) and electrophoretic light scattering (ELS) analyzer combined with non-invasive backscatter technology (NIBS) (Malvern Zetasizer Ultra, Malvern Instruments, U.K). The informed ζ-potential values correspond to the apparent ζ-potential calculated with the Helmholtz-Smoluchowski approximation. All reported values were the average result of three consecutive measurements at 25° C. on the same sample using the Zetasizer Software, 7 days after vesicle production (expect for DC-Chol containing Vs that need 2 months to stabilize). Size data was based on intensity size-distribution and corresponds to z-average between the three measurements. In some cases, to ensure the robustness of the results 3 replicates of the same formulation have been produced (specified in the corresponding Tables). In these cases, size data corresponds to z-average±standard deviation between the three replicates.

### ii) Morphology Characterization by Cryogenic Transmission Electron Microscopy (Cryo-TEM)

Formulations were examined by cryo-TEM to directly analyze vesicles morphology and study their uniformity (heterogeneity) and coexistence of structures. Information about size, shape, number of bilayers and bilayers distribution was gathered. Vitrified specimens were prepared some days after the production of the Vs. Samples were vitrified in a controlled specimen preparation chamber following well established procedures and examined in a T12 G2 Tecnai (FEI) and a Talos F200C (Thermo Fisher) microscopes at cryogenic temperatures. Perforated Ted Pella grids were used; vitrified specimens' temperature was always kept below −170° C. Images were recorded with a Gatan UltraScan 2k×2k CCD camera or a Ceta camera at low dose operation. Images were recorded at various magnifications (from 8.8 K to 53 K) to properly capture all structures, namely, at different length scales, ranging from few nm to few hundreds. No image processing was applied except for background subtraction.

### 1.2 Results

### 1.2.1 Synthesis of Vs: Effect of Surfactant Alkyl Chain Length

Vesicles comprising a phytosterol, Sit, in combination with glucose-derived surfactants, were prepared using DELOS-susp methodology. The effect of surfactants of different carbon chain length (pure lauryl glucoside (LGL-p), pure decyl glucoside (DGL-p) and pure octyl-glucoside (OGL-p)) was compared by keeping constant the glucose polar head of the surfactant. Compositions containing a mixture of OGL-p and DGL-p, as well as compositions containing a mixture of C8-C16 alkyl glucosides were also compared.

As shown in Table 1, Vs were formed only if the surfactant carbon chain length was of 12 carbons or over. The best conditions found for the synthesis of Sit: LGL-p (1:1) were used to evaluate the formation of Sit: DGL-p and Sit: OGL-p. However, no Vs were obtained in the process. Regarding the use of DGL-p and OGL-p, again no Vs were formed either when the aqueous phase was heated up to 60° C. during depressurization step, neither when additives were used to stabilize such formation, i.e., after addition of 3% w/w of the anionic additives LGC or SLS, see samples #4-6 and #9 in Table 1.

Furthermore, when a sugar-based surfactant containing a mixture of alkyl glucosides of C8-10 chains (DGL-mc₈₋₁₀, see sample #7 in Table 1) was used, no Vs were obtained either. However, when a sugar-based surfactant containing a mixture of C₈-C₁₆ alkyl glucosides (DGL-mc₈₋₁₆) was used, phytosterol-based Vs were formed (see sample #8 in Table 1).

These results confirm that not only the surfactant structure plays an important role in the formation of phytosterol-based Vs, but also the sterol side chain influences the self-assembly of the membrane components.

TABLE 1
Effect of alkyl chain length on the formation of Vs containing
phytosterols and glucose-derived surfactants.

| Sample | Surfactant | Additive | Sit:Surf:Additive | Vs |
|---|---|---|---|---|
| #1 ^{†} | LGL-p | — | 1:1:0 | Yes |
| #2 | LGL-p | LGC* | 1:1:0.05 | Yes |
| #3 | LGL-p | SLS | 1:1:0.08 | Yes |
| #4 ^{†} | DGL-p | — | 1:1:0 | No |
| #5 | DGL-p | LGC | 1:1:0.05 | No |
| #6 | DGL-p | SLS | 1:1:0.08 | No |
| #7 | DGL-m_{C8-C10} | — | 1:1:0 | No |
| #8 | DGL-m_{C8-C16} | — | 1:1:0 | Yes |
| #9 ^{†} | OGL-p | — | 1:1:0 | No |

^{†} Depressurized with the aqueous phase at 60° C.
*This reagent is a mixture of LGL, intended as alkyl glucosides of C₁₀₋₁₆ chain length, and the corresponding carboxylates; thus, the ratio 1:1:0.05 refers to the mixture and not the real content of LGC.

### 1.2.2 Synthesis of Vs: Effect of Methodology in the Formation of Vs: Comparison Between DELOS-Susp and LGL Film Hydration (TFH)

Vesicles containing APGs have previously been obtained (Muzzalupo et al. 2013) by combining either OGL-p, DGL-p or LGL-p with cholesterol using TFH methodology. However, as shown above (Example 1.2.1), it is disclosed herein that Vs containing phytosterols instead of cholesterol only form in combination with sugar-based surfactants with a carbon chain length of 12 carbons or over (e.g., LGL-p). Therefore, these results suggest that substitution of Chol by Sit may have an unexpected impact in the self-assembly of the membrane components.

To ensure that these unexpected results are independent from the methodology used for the formation of Vs, an additional study was performed to confirm the relation between the Vs formation with the surfactant carbon chain length and the sterol using TFH (as described by Muzzalupo et al. 2013). The resulting outcome was then compared to the results obtained by DELOS-susp methodology (shown in Example 1.2.1). Again, the three different surfactants OGL-p, DGL-p and LGL-p were used to evaluate the formation of Vs with Sit.

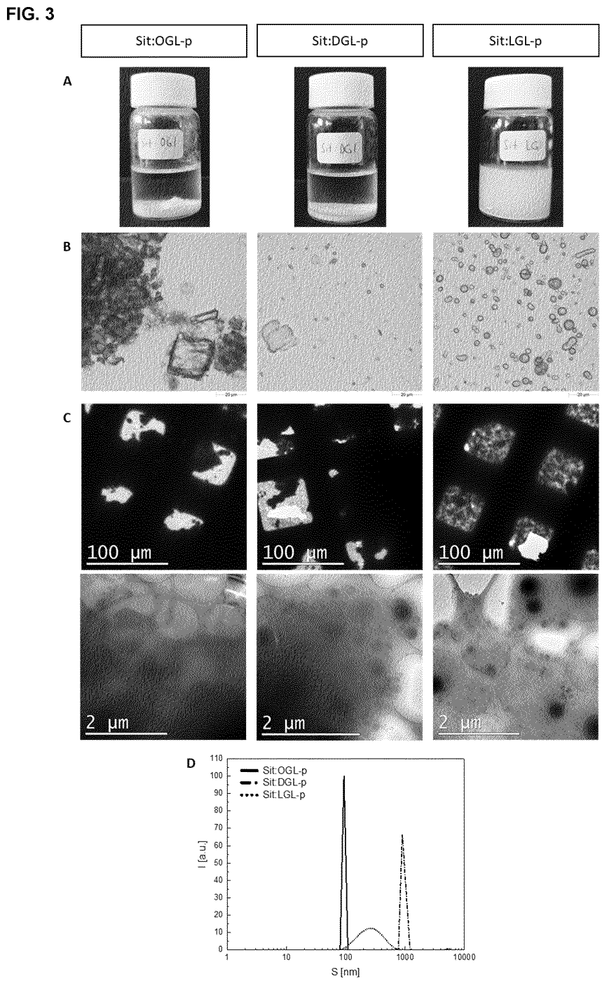

The macroscopic appearance of the three samples clearly shows a difference between the use of OGL-p and DGL-p compared to the use of LGL-p, as shown in FIG. 3(A). Sit: LGL-p (1:1) Vs showed an homogeneous dispersion, while Sit/OGL-p (1:1) and Sit/DGL-p (1:1) batches appear completely precipitate.

Further, the samples were observed with an optical microscope Morphology G3 as shown in FIG. 3 (B), showing different types of particles. In the case of Sit: OGL-p systems, the precipitate was an aggregate with crystalline aspect. In the case of Sit: DGL-p systems, two different populations of particles were observed: one having crystalline appearance and another of spheric particles. Finally, in the case of Sit: LGL-p systems, only the spheric particles population was observed, and there were no crystalline precipitates in the sample.

The sample were then observed using cryo-TEM as shown in FIG. 3(C), top panel). Sit/OGL-p and Sit/DGL-p samples clearly presented big aggregates that covered the grid quadrants where the samples were analyzed. On the other hand, the Sit/LGL-p system presented a homogeneous distribution over the grid. These results are consistent with their macroscopic appearance, as the samples which appeared precipitated (Sit/DGL-p and Sit/OGL-p) presented big aggregates which were impossible to analyze by cryo-TEM, whereas Sit/LGL-p which was homogeneous at macroscopic scale also presented a homogeneous aspect in the nanometric scale.

When analyzing the samples at a higher resolution using cryo-TEM as shown in FIG. 3(C), bottom panel, only Sit/LGL-p systems could be analyzed, as the big aggregates of Sit/DGL-p and Sit/OGL-p systems did not allow for this analysis. Regarding Sit/LGL-p systems, a large amount of Vs were observed and all of them were homogeneously distributed and not agglomerated. These results are in accordance with the previously discussed macroscopical appearance of the samples, where the agglomeration of Sit/DGL-p Vs formed unstable aggregates that sedimented, whereas the Sit/LGL-p Vs remained in suspension as they were homogeneously distributed.

Finally, the samples were analyzed by Dynamic Light Scattering (DLS) with a Zetasizer Ultra equipment as shown in FIG. 3(D). In the case of Sit/OGL-p (particle size 4041 nm and Pdl 1.60) and

Sit/DGL-p (particle size 8376 nm and Pdl 1.61), the samples were so heterogeneous that the measures did not pass the quality criteria of the equipment, meaning that the data was not reliable. On the other hand, Sit/LGL-p Vs showed particle sizes of 250 nm and Pdl 0.23+0.06, which was considered reliable data.

These results confirm that the synthesis of Vs comprising phytosterols is dependent on the surfactant alkyl chain length, regardless of the methodology used. Vs containing for e.g., Sit can only be formed when APGs of 12 carbons or over are used. The use of OGL or DGL does not allow for the formation of phytosterols-containing Vs, unlike Vs comprising cholesterol, as described in Muzzalupo et al. 2013 (see Table 2). Therefore, it appears clear that surprisingly, the substitution of cholesterol for phytosterols does have an impact on the relation between the APG used and the successful formation of Vs, regardless of the methodology used.

TABLE 2
Comparison of Sit/APGs vs. Chol/APGs structures
particle size and Pdl determined by DLS at time.

| | Sit/APGs structures | | | Chol/APGs Vs (Muzzalupo et al.) | | |
|---|---|---|---|---|---|---|
| Parameter | Sit:OGL | Sit:DGL | Sit:LGL | Chol:OGL | Chol:DGL | Chol:LGL |
| Particle size (nm) | 4071 | 8376 | 250 | 292 | 345 | 515 |
| Pdl | 1.60 | 1.61 | 0.23 | 0.21 | 0.21 | 0.005 |

### 1.2.3 Synthesis of Sit: LGL Vs

Taking into consideration the results of Examples 1.2.1 and 1.2.2, different vesicular systems were prepared applying the DELOS-susp methodology, which comprise phytosterols comprising ß-sitosterol and LGL as a glucose-derived surfactant.

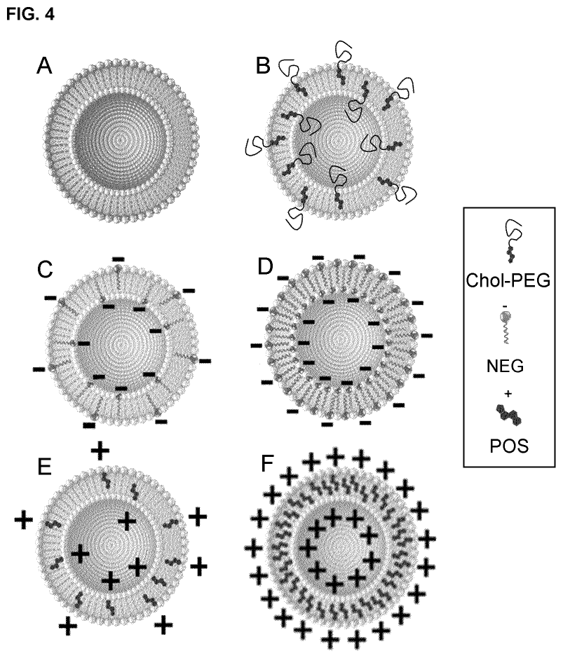

The Vs formed by DELOS-susp methodology include neutral, positive, and negative delivery systems which were formed mainly by self-assembly of Sit and LGL (see Table 3 and FIG. 4).

The neutral carriers were large unilamellar vesicles with particles sizes around 217-683 nm and colloidal stabilities ranging from weeks to more than 12 months depending on the purity of the APG surfactant used. Negative and positively charged carriers, based on glucose-derived surfactants, were both small unilamellar vesicles. Negatively charged carriers, composed of phytosterols, LGL and LGC, ranged from 148 to 193 nm depending on LGC surfactant molar ratio and they maintained its physicochemical properties for more than 1 year. On the other hand, positive Vs were obtained with the incorporation of different molar ratio of DC-Cholesterol (DC-Chol). These Vs sizes ranged from 64 to 104 nm and they are stable for more than 12 months.

TABLE 3
Summary of composition and physicochemical properties of
the new Vs platform obtained by DELOS technology (n = 3).

| | Composition | Size | | ζ-Pot |
|---|---|---|---|---|
| Type of NV | (molar ratio) | [nm] | Pdl | [mV] |
| Neutral | Sit:LGL- | 217 ± 11 | 0.22 ± 0.01 | −42 ± 3 |
| | m_{C12-16} (1:1) | | | |
| | Sit:LGL- | 683 ± 45 | 0.43 ± 0.11 | −35 ± 2 |
| | p (1:1) | | | |
| | Sit:LGL- | 134 ± 9 | 0.18 ± 0.05 | 2 ± 6 |
| | p:Chol-PEG₆₀₀ | | | |
| | (0.94:1:0.06) | | | |
| | Sit:LGL- | 193 ± 4 | 0.24 ± 0.03 | −51 ± 2 |
| | p:LGC (1:1:0.05) | | | |
| | Sit:LGL- | 166 ± 2 | 0.20 ± 0.04 | −41 ± 7 |
| | p:SL (1:1:0.1) | | | |
| | Sit:LGL- | 186 ± 10 | 0.22 ± 0.03 | −55 ± 3 |
| | p:SLS (1:1:0.08) | | | |
| Negative | Sit:LGL- | 182 ± 9 | 0.22 ± 0.01 | −61 ± 2 |
| | p:LGC (1:0.8:0.2) | | | |
| | Sit:LGL- | 148 ± 4 | 0.20 ± 0.01 | −52 ± 5 |
| | p:LGC (1:0.5:0.5)j | | | |
| | Sit:LGL- | 153 ± 10 | 0.18 ± 0.01 | −64 ± 7 |
| | p:LGC (1:0.2:0.8) | | | |
| | Sit:LGL- | 161 ± 8 | 0.18 ± 0.05 | −70 ± 5 |
| | p:LGC (1:0:1) | | | |
| Positive | Sit:LGL- | 104 ± 13 | 0.18 ± 0.04 | 55 ± 3 |
| | p:DC-Chol | | | |
| | (1:1:0.05) | | | |
| | Sit:LGL- | 97 ± 5 | 0.11 ± 0.02 | 72 ± 15 |
| | p:DC-Chol | | | |
| | (0.8:1:0.2) | | | |
| | Sit:LGL- | 72 ± 8 | 0.11 ± 0.02 | 75 ± 16 |
| | p:DC-Chol | | | |
| | (0.5:1:0.5) | | | |
| | Sit:LGL- | 64 ± 10 | 0.13 ± 0.02 | 72 ± 17 |
| | p:DC-Chol | | | |
| | (0.2:1:0.8) | | | |
| | Sit:LGL- | 70 ± 12 | 0.17 ± 0.05 | 84 ± 14 |
| | p:DC-Chol | | | |
| | (0:1:1) | | | |

### 1.2.4 Synthesis of Neutral Vs Comprising Sit/LGL

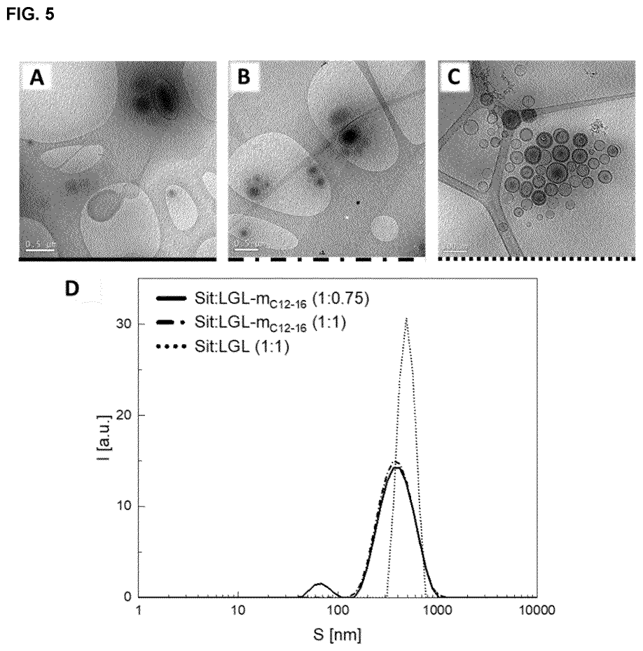

To evaluate the formation of neutral Vs, LGL molecules of different quality and purity were used. When using cosmetic grade reagents containing a mixture of alkyl glucosides of different carbon chain length (e.g., LGL-mc12-16) and known to be of lower purity, Vs were formed in any of the tested Sit: LGL molar ratios. However, other structures like ribbons, big MLVs and solid particles were also observed, according to cryo-TEM images and particle size measurements of the best performance DELOS samples, as shown in FIG. 5(A-B).

In those lines, the formation of Vs using 98% purity LGL (LGL-p) was also evaluated. Neutral Sit: LGL-p (1:1) Vs were obtained when the aqueous phase temperature was increased to 60° C. during the depressurization step, as shown in FIG. 5(C). Neutral Sit/LGL Vs produced with LGL-p were bigger and more polydisperse (particle size 683±45 nm and Pdl 0.43±0.11) than those produced with the mixture of APGs (particle size 217±11 nm and Pdl 0.22±0.01) (see FIG. 5(D).

### 1.2.5 Stabilization of Neutral Vs by Adding Chol-PEG600

Pegylated cholesterol was used to increase colloidal stability of neutral Sit/LGL-p carriers. Pegylated cholesterol (Chol-PEG600) at 7.4% w/w was added to the membrane of neutral Vs.

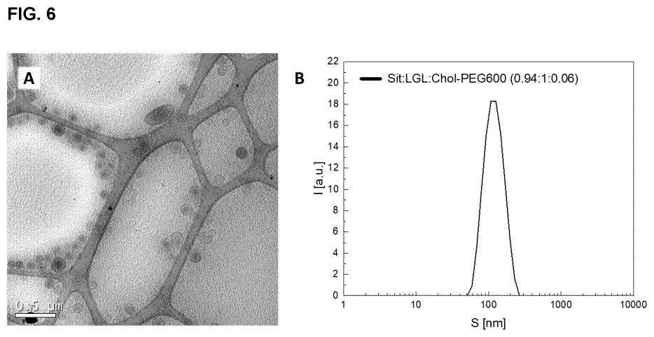

The stability of Vs was increased to over 3 months as shown in FIG. 6. Remarkably, such additive avoids the use of high temperatures during the Vs formation, which is a crucial condition for the encapsulation of thermolabile active molecules such as proteins. Pegylation of Vs membranes can help enhance Vs' colloidal stability due to the steric hindrance caused by the long carbon chains of PEG molecules and by forming hydrogen bonds with the solvent, thus avoiding the agglomeration of the vesicles.

### 1.2.6 Synthesis of Positively and Negatively Charged Vs Comprising Sit/LGL

The synthesis of charged Vs was achieved by adding charged molecules, both anions and cations, to the membrane of neutral Vs, which resulted in an improvement of the colloidal stability by charge repulsion.

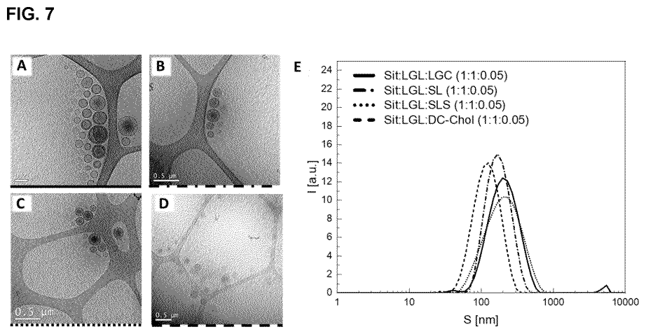

A 3% w/w of a charged molecule was added to the membrane components. Firstly, anionic surfactants such as LGC, SL and SLS, which come from lauryl alcohol of C₁₂ chain length, allowed for Vs formation, as shown in FIG. 7(A-C). Secondly, DC-Chol, which is a sterol-like molecule, was added as a cationic additive to the Sit/LGL membrane, and also allowed for Vs formation, as shown in FIG. 7(D). The resulting charged Vs showed a high stability of up to over 12 months. Remarkably, such additives avoid the need of high temperatures during the Vs formation, which is a crucial condition for the encapsulation of thermolabile active molecules such as proteins.

These results show the capacity of this new sugar-based Vs platform to form both positively and negatively charged Vs and therefore confirm the high number of potential applications resulting from this platform. For instance, DC-Chol is a pH sensitive molecule that may be useful for intracellular drug delivery.

### 1.2.7 Screening of LGL: LGC and Sit: DC-Chol Ratios

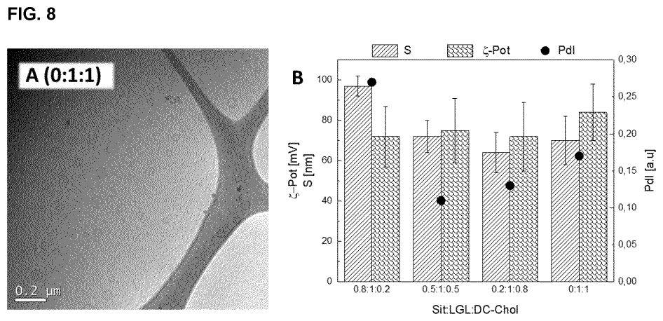

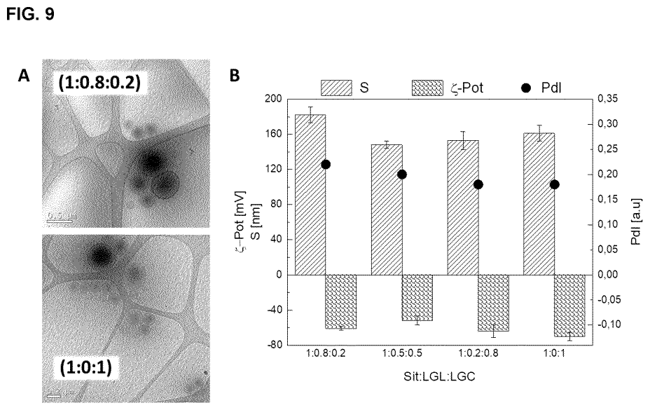

The versatility of the new sugar-based Vs platform was further explored by screening different ratios of the charged membrane component until the complete replacement of Sit by DC-Chol and LGL-p by LGC in each case, as shown in FIG. 8 and FIG. 9, respectively. The average size of the Vs decreased from 104 to 64 nm as DC-Chol ratio increased, whereas the opposite behavior was observed with the ζ-potential since the higher the amount of positively charged surfactant, the greater the surface charge of the particle. A similar behavior was observed when different quantities of LGC were screened (see FIG. 9). In both cases, the greatest change in particle size was observed between LGL-p: LGC or Sit: DC-Chol (0.8:0.2) and (0.5:0.5) molar ratios, while increasing LGC or DC-Chol ratio over 0.5 did not generate significant changes in the physicochemical properties of the Vs. Regarding the ζ-potential, no significant changes were observed between the Vs containing different molar ratio of LGC and DC-Chol; however, the ζ-potential value is higher in absolute values respect to neutral Sit/LGL Vs.

### Example 2. Encapsulation of Active Ingredients in Glucose-Derived Vs Platform

The encapsulation of different active ingredients was evaluated to demonstrate the potential applications of the Vs platform disclosed herein (e.g., pharmaceutical and cosmetic applications). A number of molecules/compounds representing three different types of actives including small biomolecules, proteins and dyes were selected to be screened in negative and positive LGL vesicular systems, in accordance with the physicochemical properties of the molecules to be encapsulated.

### 2.1. Materials and Methods

### Materials

The materials used to synthesize the Vs of the present invention were as disclosed in Section 1.1 Materials of EXAMPLE 1. Further, additional materials which correspond to the active ingredients used were added. These are as follows: 7-dehydrocholesterol (7-DHC), Methotrexate (MTX) and 2-O-α-D-Glucopyranosyl-L-ascorbic Acid (ascorbyl glucoside, AG) were purchased from Merck. 1,1′-Dioctadecyl-3,3,3′,3′-Tetramethylindodicarbocyanine, 4-Chlorobenzenesulfonate salt (DiD) and 1,1′-Dioctadecyl-3,3,3′,3′-Tetramethylindocarbocyanine perchlorate (Dil) were obtained from Invitrogen, Thermo Fisher Scientific. Niacinamide PC (NCA) and DL-alpha-Tocopherol (TCP) were from DSM. Pure cannabidiol (CBD) Isolate was obtained from CBD Cure. PnP b-FGF (basic fibroblast growth factor was obtained from PnP Biopharm. Human growth hormone (HGH) was purchased from Prospec. Ethanol HPLC grade was purchased from Scharlab.

### DELOS-Susp Methodology

### i) Synthesis of Vs Containing Active Ingredients

The synthesis of Vs was performed as disclosed in 1.1 DELOS-susp Methodology section of EXAMPLE 1. Further, active ingredients were added in certain steps of the process depending on their characteristics. Lipophilic actives (7-dehydrocholesterol, tocopherol, cannabidiol, Dil and DiD) are added during step (a) alongside with the membrane components (at atmospheric pressure in the vessel). Hydrophilic actives (niacinamide, ascorbyl glucoside, methotrexate, b-FGF and HGH) were added during step (b) in the aqueous solution containing the water-soluble surfactants.

### ii) Entrapment Efficiency (EE) by UV-Vis Spectroscopy

Entrapment efficiency (EE) of each active was determined by UV-Vis absorbance using an UV-Vis spectrophotometer (Thermo Scientific™ Evolution™ 201 UV-Visible Spectrophotometers, Thermo Scientific). For lipophilic actives (7-DHC, CBD, TCP, DiD and Dil) Vs membrane was dissociated to release the active by diluting the sample in ethanol at 80% (v/v) until a value of absorption below 1 and inside the calibration curve obtained. The concentration of each active was determined using a calibration curve, using the same media than the corresponding Vs system. The active was quantified before and after filtering process and both values were used to calculate the EE using Equation 1 (as described herein). As the lipophilic molecules are not soluble in aqueous media, it was considered that the filtered sample contained only the integrated active, since all the precipitated and non-integrated active should remain in the filter.

$${EE}_{lip ophilic actives} ( \% ) = \frac{{[ active ]}_{filtered sample}}{{[ active ]}_{non - filtered sample}} * 100 \qquad Equation 1$$

A different protocol was followed for hydrophilic actives (NCA, AG and MTX), in which, after the Vs filtration process, another step was applied to separate the non-integrated active. For NCA and AG, an ultracentrifugation step of the filtered sample was performed (6h, 4° C., 600000g) (Sorvall Discovery M150 Micro-Ultracentrifuge, Thermo Scientific). In the case of MTX, the separation of the non-integrated active from the Vs was done by Tangential Flow Filtration (TFF) (KrosFlo® Research Ili TFF diafiltration system (KR2i), with a mPES filter hollow column cut-off 300 KDa (C04-E100-05-N, Spectrum Labs, SL). The supernatant in the case of centrifugated samples and the permeate fraction in the case of filtered by TFF ones was recovered and diluted until a value of absorption below 1 and inside the calibration curve. The concentration was calculated using a calibration curve using the same media of the corresponding Vs system. The entrapment efficiency was then calculated using Equation 2, as described herein:

$${EE}_{hydrophilic actives} ( \% ) = \frac{{[ active ]}_{total theoretical} - {[ active ]}_{supernatant / permeate fraction}}{{[ active ]}_{total theoretical}} * 100 \qquad Equation 2$$

iii) Entrapment efficiency (EE) by fluorescence spectroscopy: Entrapment efficiency (EE) of both proteins was determined by fluorescence spectroscopy using a Varian Cary Eclipse (Agilent Technolo-gies, Santa Clara, USA). In both cases, the separation of the non-integrated protein from that integrated in Vs was done by TFF (KrosFlo® Research 11i TFF diafiltration system (KR2i), with a mPES filter hollow column cut-off 300 KDa (C04-E100-05-N, Spectrum Labs, SL). Then, the permeate fraction was recovered and analyzed by fluorescence, exciting the proteins at 278 nm. The concentration was calculated using a calibration curve using the same media of the corresponding Vs system. The entrapment efficiency was then calculated using Equation 2, as described herein.

### 2.2 Results

### 2.2.1 Sit: LGC and Sit: LGL: LGC Systems

The Sit: LGC (1:1) system was used to integrate both lipophilic and hydrophilic actives, whereas Sit: LGL: LGC (1:0.8:0.2) was selected to test encapsulation of only lipophilic molecules, as shown in

Table 4. The following molecules were used:
Lipophilic small molecules: 7-dehydrocholesterol (7DHC), a photochemically converted to vitamin D₃ in the skin, cannabidiol (CBD), a phytocannabinoid contained in cannabis plants with many medical uses under study, α-Tocopherol (TCP), a type of vitamin E, and carbocyanine dyes (Dil and DiD) as fluorescent markers.
Hydrophilic vitamins: niacinamide (NCA) or vitamin B3, and ascorbyl glucoside (AG), a vitamin C derivative.
Small protein: basic Fibroblast Growth Factor (b-FGF)

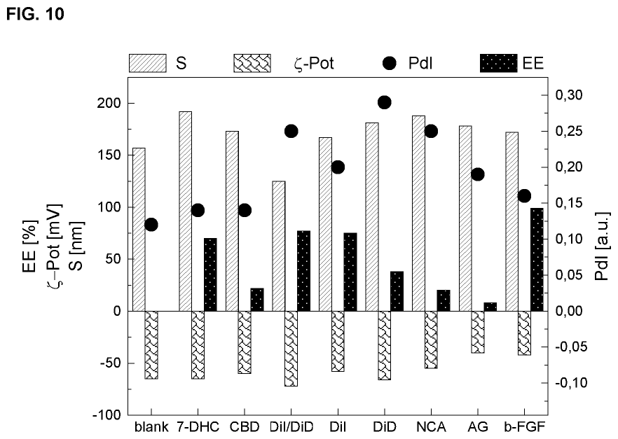

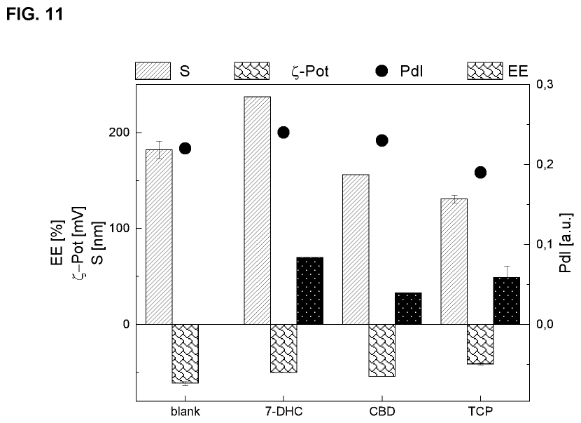

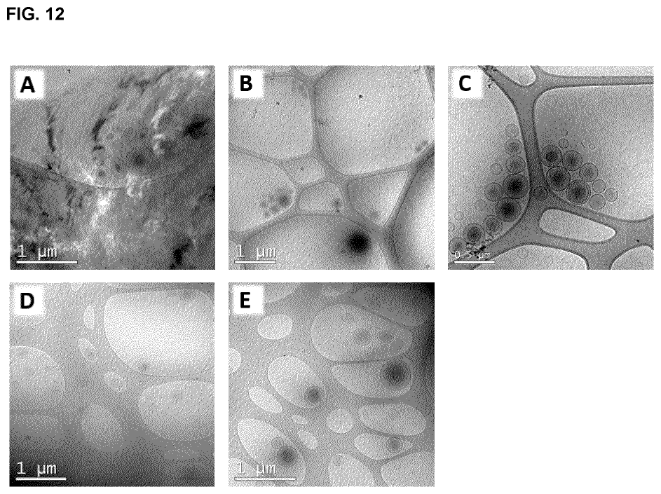

Both systems (Sit: LGC and Sit: LGL: LGC) showed a high encapsulation efficiency (EE) of lipophilic actives (49-77%), whereas Sit: LGC showed a lower EE for the hydrophilic actives (8-20%), except for CBD (22-33%) (Table 4, FIG. 10-12). This is consistent with the fact that lipophilic actives can be inserted in the Vs membrane, which is also lipophilic, whereas hydrophilic actives are less likely to integrate in the aqueous lumen of the Vs. In the case of b-FGF it was highly efficiently encapsulated (>99%) in Sit/LGC carriers due to the electrostatic interactions between the positively charged amino acids of the protein and the negatively charged Vs at working pH.

TABLE 4
Physicochemical characterization and entrapment efficiency (EE)
of active-loaded sugar based systems Sit:LGC and Sit:LGL:LGC Vs.

| | | | | Theoretical | | | | |
|---|---|---|---|---|---|---|---|---|
| | | Molar | Active | Active loading | EE | Size | | ζ-Pot |
| Sample | Composition | ratio | compound | [mg/mL] | [%] | [nm] | Pdl | [mV] |
| #1 | Sit:LGC | 1:1 | 7DHC | 0.48 | 70 | 192 | 0.14 | −65 |
| #2 | Sit:LGC | 1:1 | CBD | 1.25 | 22 | 173 | 0.15 | −60 |
| #3 | Sit:LGC | 1:1 | Dil/DiD | 0.040/0.045 | 78/77 | 125 | 0.25 | −72 |
| #4 | Sit:LGC | 1:1 | Dil | 0.037 | 75 | 167 | 0.20 | −58 |
| #5 | Sit:LGC | 1:1 | DiD | 0.037 | 38 | 181 | 0.29 | −66 |
| #6 | Sit:LGC | 1:1 | NCA | 1.25 | 20 | 188 | 0.25 | −55 |
| #7 | Sit:LGC | 1:1 | AG | 1.25 | 8 | 178 | 0.19 | −40 |
| #8 | Sit:LGC | 1:1 | b-FGF | 0.05 | >99 | 172 | 0.16 | −42 |
| #9 | Sit:LGL:LGC | 1:0.8:0.2 | 7DHC | 0.3 | 70 | 237 | 0.24 | −50 |
| #10 | Sit:LGL:LGC | 1:0.8:0.2 | CBD | 0.75 | 33 | 156 | 0.23 | −54 |
| #11* | Sit:LGL:LGC | 1:0.8:0.2 | TCP | 0.75 | 49 ± 12 | 131 ± 4 | 0.1 ± 0.01 | −41 ± 1 |

*Three replicates of the same formulation were done (n = 3)

### 2.2.2. Sit: LGL-p: DC-Chol System

The Sit: LGL-p: DC-Chol (0.8:1:0.2) system, which has a cationic character given by the protonated amine group of DC-Chol, was also used to integrate lipophilic and hydrophilic molecules. The chosen lipophilic actives were the following: 7-DHC, CBD and TCP. In the case of hydrophilic actives, they were selected according to their charge at the working pH, to favor an electrostatic interaction with the positive charged Vs. The methotrexate (MTX) molecule, which is a chemotherapy agent and immune-system suppressant, was selected as an example of small hydrophilic molecule, whereas the Human Growth Hormone (HGH) was selected as an example of small protein.

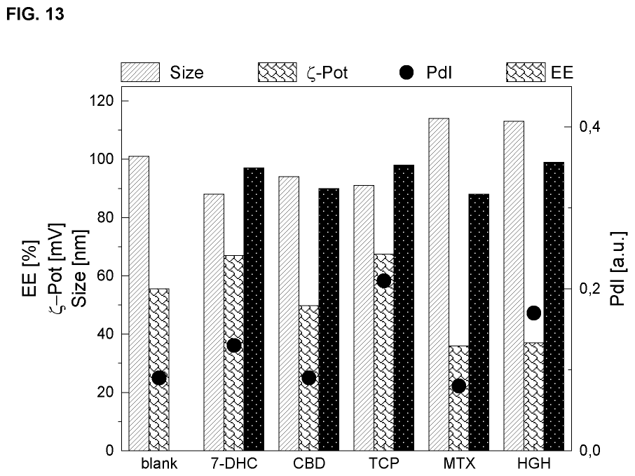

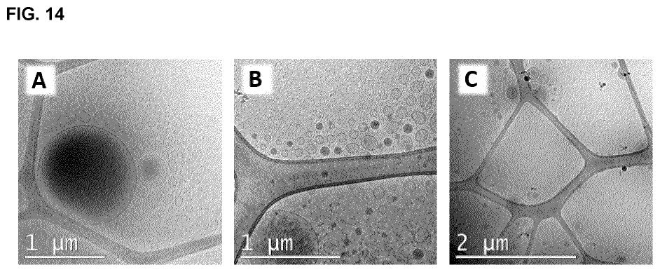

Similar results to the above-mentioned systems were obtained. The encapsulation efficiency of lipophilic actives was very high (all>90%), and even higher than the EE of negatively charged Vs. Un-expectedly, MTX was also highly efficiently encapsulated (EE=88%), despite being a hydrophilic molecule. Finally, in regard to protein HGH, it also showed an extremely high EE of over 99%. These results are shown in Table 5 and FIG. 13-14.

TABLE 5
Physicochemical characterization and entrapment efficiency (EE) of active-loaded Sit:LGL:DC-Chol Vs.

| | | | | Theoretical | | | | |
|---|---|---|---|---|---|---|---|---|
| | | Molar | | Active | EE | Size | | ζ-Pot |
| Sample | Composition | ratio | Active | [mg/mL] | [%] | [nm] | Pdl | [mV] |
| #12 | Sit:LGL-p:DC-Chol | 0.8:1:0.2 | 7DHC | 0.28 | 97 | 88 | 0.13 | 67 |
| #13 | Sit:LGL-p:DC-Chol | 0.8:1:0.2 | CBD | 0.70 | 90 | 94 | 0.09 | 50 |
| #14* | Sit:LGL-p:DC-Chol | 0.8:1:0.2 | TCP | 0.70 | 98 ± 2 | 93 ± 2 | 0.18 ± 0.03 | 62 ± 5 |
| #15 | Sit:LGL-p:DC-Chol | 0.8:1:0.2 | MTX | 0.26 | 88 | 114 | 0.08 | 36 |
| #16 | Sit:LGL-p:DC-Chol | 0.8:1:0.2 | HGH | 0.002 | >99 | 113 | 0.17 | 37 |

*Three replicates of the same formulation were done (n = 3)

### Example 3. Functional Tests of Active Ingredients Integrated in Glucose-Derived Vs Platform

After demonstration of active ingredients encapsulation, their functional activity after integration in Vs was evaluated. It was important to prove the conservation of functionality after formulation. Two different actives were tested using the same vesicular system and a specific assay was performed for each to study their particular functionalities.

### 3.1. Materials and Methods

### Materials

The materials used to synthesize the Vs were as disclosed in Section 1.1 Materials of EXAMPLE 1 and Section 2.1 Materials of EXAMPLE 2. Further, additional materials and reagents were used to perform the functional assays: 2,2-diphenyl-1-picrylhydrazyl (DPPH) and cholecalciferol, purchased from Merck. HPLC grade solvents methanol (MeOH) was acquired from Alco (Barcelona, Spain), isopropanol (IPA) was purchased in Fischer (New Hampshire, United States) and formic acid (HCOOH) was acquired from Sigma-Aldrich (Missouri, United States).

### DELOS-Susp Methodology

### i) Synthesis of Vs Containing Active Ingredients

The synthesis of Vs was performed as disclosed in 1.1 DELOS-susp Methodology section of EXAMPLE 1 and 1.1 DELOS-susp Methodology section of EXAMPLE 2. Vs loaded with TCP were prepared with Sit, LGL-p and LGC at a molar ratio of 1:0.8:0.2 and a TCP concentration of 0.75 mg/ml (dispersant media H₂O/ETOH 15% (v (/v)). Vs loaded with 7DHC were prepared with Sit, LGL-p, LGC and 7DHC at a molar ratio 0.2:0.8:0.2:0.8.

### Functional Assay for α-Tocopherol (TCP): Antioxidant Capacity Evaluation

### i) Antioxidant Capacity by 2,2-Diphenyl-1-Picrylhydrazyl (DPPH) Assay

DPPH assay is a well-known and established method to measure antioxidant capacity of a molecule. DPPH is a liposoluble and stable free radical that is easily reduced in presence of antioxidants. When DPPH is in its oxidated form presents a violet color that changes to yellow/white when is reduced. This color change can be followed using UV-Vis spectroscopy. The assay was carried out in ethanol always maintaining a molar ratio DPPH: TCP of 1:0.3. In order to have 3 mL of final volume, DPPH was diluted in ethanol to have a final concentration of 63.4 μM. Free TCP and TCP DELOS-NVs were diluted to have a final concentration of 19 UM of TCP. Each solution was then mixed and kept at room temperature for 30 minutes in the dark. The absorbance of the solution was measured at 517 nm using a Thermo Scientific™ Evolution™ 201/220 UV-Visible spectrophotometer (Thermo Scientific). The percentage of TCP antioxidant capacity was calculated using the Equation 3, where Abs_{control} is the absorbance at 517 nm of DPPH in ethanol and Absₛₐₘₚₗₑ is the absorbance at 517 nm of the sample prepared as described (free TCP or TCP DELOS-NVs with DPPH).

$$TCP Antioxidant Capacity ( \% ) = \frac{( {Abs}_{control} - {Abs}_{sample} )}{{Abs}_{control}} * 100 \qquad Equation 3$$

Functional Assay for 7-Dehydrocholesterol (7DHC): Transformation from 7DHC to Vitamin D

### i) UV-Vis Spectroscopy Characterization Before Irradiation

The concentration of 7DHC loaded in Vs was previously characterized by UV-Vis spectroscopy as disclosed in 2.1 Materials and methods section of EXAMPLE 2.

ii) Irradiation Assay 7DHC is a molecule present in human keratinocytes of the skin that it is transformed to cholecalciferol (vitamin D) by sun irradiation. The objective of this functional test was to demonstrate that in suspension, free 7DHC and encapsulated in the new Vs they both have the same conversion ratio to cholecalciferol. To do so, Vs composed by Sit, LGL-p, LGC and 7DHC at a molar ratio 0.2:0.8:0.2:0.8 were prepared. Furthermore, a suspension of 7DHC in water/EtOH 15% (v/v) at the same concentration that the 7DHC in the Vs suspension was also prepared. Then, 1 mL of each suspension was placed in a well of a 12 well-plate (n=3). The samples were irradiated using a UV lamp (λ=302 nm) during 30 min and 1 h. Finally, the samples were incubated at room temperature for 48 h in the absence of light before being analyzed by HPLC.
iii) Determination of the Conversion of 7DHC to Vitamin D by HPLC Analysis

The concentration of 7DHC and vitamin D (cholecalciferol) after 30 min and 1 h of UV irradiation were quantified by HPLC. Results were compared with the initial concentration before irradiation measured by UV-Vis spectroscopy. Samples of free 7DHC and 7DHC loaded Vs were analyzed. Stock standard solutions of 7DHC and vitamin D were prepared in methanol and standard solutions of lower concentration were obtained by dilution of stock solutions in methanol. To prepare the samples, 1 mL of each sample was dissolved in 4 mL of methanol. A volume of 10 μL of each sample was injected into HPLC system. The analysis was carried out using a Waters liquid chromatographic system (Milford, MA, USA), connected to a Waters PDA detector type HPLC 2998. The software Empower 3 was used for instrument control and data analysis. Detection was carried out at 282 nm for 7DHC and 264 nm for cholecalciferol.

An Atlantis Premier BEH C18 AX 2.5 μm 4.6×150 mm (Waters) was used to separate sample components before detection. The column temperature was set to 40° C. Two solvents, A: water and B: methanol-isopropyl alcohol-formic acid (94.9:5:0.1, v: v: v), were used in an isocratic elution mode. Solvent B was vacuum-filtered through a PVDF membrane (0.45 μm pore diameter). The mobile phase flow rate was 2 ml min−1. An isocratic flow of 10% of mobile phase A+90% of mobile phase B mode was used, where the total running time was of 23 minutes. Quantification was performed by integration of the peak area of the corresponding analyte and interpolation of the peak area in 7-DHC or cholecalciferol standard curves.

### 3.2 Results

### 3.2.1 Functional Assay for TCP: Antioxidant Capacity Evaluation

The antioxidant capacity of TCP was evaluated once it was integrated in the NVs to verify that the molecule was still active after the encapsulation. The antioxidant capacity was measured by the 2,2-diphenyl-1-picrylhydrazyl (DPPH) assay. DPPH is a stable free radical that can be reduced in the presence of hydrogen-donating antioxidants such as TCP. The activity of TCP after encapsulation was compared with free TCP dissolved in ethanol. The assay was performed in a molar ratio DPPH: TCP of 1:0.3. Considering that 1 mol of TCP reduces 2 mol of DPPH, the antioxidant capacity expected in the assay was 60%. After the analysis, both samples, free TCP and TCP encapsulated in DELOS-NVs had the expected antioxidant (OX) capacity, showing that the encapsulation of TCP in the Vs did not affect the AOX functionality of the molecule (Table 6). Thus, it can be concluded that a lipophilic vitamin can be loaded in the Vs, that is an aqueous formulation, maintaining intact its antioxidant capacity.

TABLE 6
Antioxidant capacity of free TCP and DELOS-NVs
containing TCP (TCP DELOS-NV) calculated by DPPH
method. Values represent the antioxidant capacity
mean ± s.d. of two independent experiments.

| | Free | TCP loaded |
|---|---|---|
| | TCP | DELOS-NV |
| Antioxidant | 60 ± 9 | 63 ± 5 |
| capacity (%) | | |

### 3.2.2 Functional Assay for 7DHC: Conversion to Vitamin D

To assess if 7DHC can be converted to vitamin D (VitD) after integration of the molecule in Vs, irradiation assays were performed. Samples of 7DHC free and 7DHC loaded in Vs, both in water/EtOH 15% (v/v), were irradiated using a UV lamp (λ=302 nm) during 30 min or 1 h (n=3). Conversion of 7DHC to VitD was quantified by HPLC. A decrease on 7-DHC concentration in the irradiated samples was expected, while VitD was expected to appear in the samples, indicating that 7DHC is being converted to VitD.

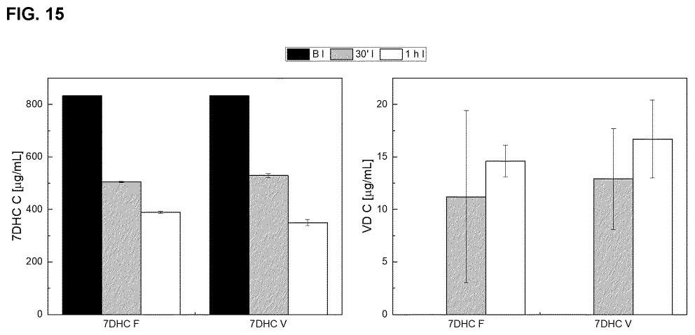

As seen in FIG. 15 and Table 7, either for 7DHC free or loaded in Vs there is a decrease in 7DHC concentration. This reduction was similar in both cases (after 1 h from 832 μg/mL to 389±4 μg/mL and 350±12 μg/mL respectively), indicating that the loading of 7DHC in the vesicles did not affect its conversion to VitD. Regarding VitD production, the levels after 1 h were also similar between the sample with 7DHC free and loaded in the Vs (15±1 μg/mL and 17±4 μg/mL). Due to these results, it can be concluded that after encapsulation, 7DHC maintains its capacity to transform to vitamin D, which is a molecule with an active role in cell functionality.

TABLE 7
7DHC and VitD quantification by HPLC after irradiation
of 7DHC loaded vesicles and suspensions of free, non-
encapsulated 7DHC. Values represent the molecule concentration
mean ± RSD (%) of three independent experiments.

| | 7DHC | | VitD | |
|---|---|---|---|---|
| | concen- | | concen- | |
| | tration | RSD | tration | RSD |
| Sample | (μg/mL) | (%) | (μg/mL) | (%) |
| 7DHC free | | | | |
| (non encapsulated) | | | | |
| 30 min irradiation | 505 | 2 | 11 | 8 |
| 1 h irradiation | 389 | 4 | 15 | 2 |
| 7DHC loaded NVs | | | | |
| 30 min irradiation | 529 | 7 | 13 | 5 |
| 1 h irradiation | 350 | 12 | 17 | 4 |

### REFERENCES

### Non-Patent Literature

Salim, M., Abou-Zied, O. K., Udani Kulathunga, H., Baskaran, A., Kuppusamy, U. R. & Hashim, R. (2015). Alkyl Mono- and Di-Glucoside Sugar Vesicles as Potential Drug Delivery Vehicles: Detecting Drug Release Using Fluorescence. RSC Adv., 5 (68), 55536-55543.
Muzzalupo, R., Tavano, L. & La Mesa, C. (2013). Alkyl Glucopyranoside-Based Niosomes Containing Methotrexate for Pharmaceutical Applications: Evaluation of Physico-Chemical and Biological Properties. Int. J. Pharm., 458 (1), 224-229.
Manconi, M., Vila, A. O., Sinico, C., Figueruelo, J., Molina, F. & Fadda, A. M. (2006). Theoretical and Experimental Evaluation of Decypolyglucoside Vesicles as Potential Drug Delivery Systems. J. Drug Deliv. Sci. Technol., 16 (2), 141-146.
Manconi, M., Sinico, C., Valenti, D., Lai, F. & Fadda, A. M. (2006). Niosomes as Carriers for Tretinoin: III. A Study into the in Vitro Cutaneous Delivery of Vesicle-Incorporated Tretinoin. Int. J. Pharm., 311 (1-2), 11-19.
Plaza-Oliver, M., Santander-Ortega, M. J., Lozano, M. V. (2021). Current approaches in lipid-based nanocarriers for oral drug delivery, Drug Deliv. Transl. Res. 11 471-497.

### PATENT LITERATURE

WO 2006/079889 A1

What is claimed is:

1. A vesicle comprising at least one phytosterol and at least one glucose-derived surfactant.

2. The vesicle according to claim 1, wherein the at least one glucose-derived surfactant is an alkyl polyglucoside surfactant or a derivative thereof, wherein the alkyl polyglucoside surfactant or the derivative thereof has a carbon chain length of at least twelve carbons.

3. The vesicle according to claim 2, wherein the alkyl polyglucoside surfactant or the derivative thereof is selected from the group consisting of octyl glucoside, decyl glucoside, lauryl glucoside, tetradecyl glucoside, hexadecyl glucoside, tetradecyl D-glucoside, hexadecyl β-D-glucopyranoside, octadecyl D-glucoside, arachidyl glucoside, a mixture of C12-20 alkyl glucoside, cetearyl glucoside, C20-22 alkyl glucoside, coco-glucoside, isostearyl glucoside, ocyldodecyl glucoside, phosphorus derivatives of alkyl polyglucoside and lauryl glucoside carboxylate.

4. The vesicle according to claim 3, wherein the alkyl polyglucoside surfactant is lauryl glucoside, tetradecyl glucoside, hexadecyl glucoside or a combination thereof.

5. The vesicle according to claim 4, wherein the alkyl polyglucoside surfactant is lauryl glucoside.

6. The vesicle according to claim 1, wherein the phytosterol comprises β-sitosterol.

7. The vesicle according to claim 1, wherein the vesicle further comprises a stabilizing agent which is selected from the group consisting of a neutral additive, a cationic additive, and an anionic additive.

8. The vesicle according to claim 7, wherein the stabilizing agent is a neutral additive which is a conjugate comprising a cholesterol moiety and a polyethylene glycol moiety which are covalently attached.

9. The vesicle according to claim 7, wherein the stabilizing agent is an anionic additive which is selected from the group consisting of lauryl glucose carboxylate, sodium lauroyl sarcosinate and sodium laurate, and combinations thereof.

10. The vesicle according to claim 7, wherein the stabilizing agent is a cationic additive which is DC-cholesterol.

11. The vesicle according to claim 1, wherein the vesicle further comprises an active ingredient, which is selected from the group consisting of a pharmaceutical agent, a cosmetic agent, a food additive or food supplement, a vitamin, a diagnosing agent and a detecting agent.

12. (canceled)

13. A composition for diagnosing or detecting a condition comprising the vesicles of claim 1 and at least one excipient, vehicle or carrier.

14. A suspension comprising the composition as defined in claim 13, for the manufacture of a suspension for topical, oral, intravenous, inhalation, parenteral, mucosal, subcutaneous, or ocular administration.

15. A composition comprising a plurality of vesicles as defined in claim 1, wherein the composition is selected from the group consisting of a pharmaceutical composition, a cosmetic composition, a nutraceutical composition, a food composition, a food additive or food supplement and a detecting or diagnostic composition.

16. A method to prevent and/or treat a disease in a subject in need thereof, comprising administering to the subject a therapeutically effective amount of the vesicle according to claim 1.

17. A delivery system comprising the vesicles of claim 1 and at least one excipient, vehicle or carrier.
